(19) United States
(12) Patent Application Publication
(10) Pub. No.: US 2004/0043040 A1
(43) Pub. Date: Mar. 4, 2004

(54) SDF-1 beta tumor vaccines and uses therefor

(76) Inventor: Kyriaki Dunussi-Joannopoulos, Belmont, MA (US)

Correspondence Address: MINTZ, LEVIN, COHN, FERRIS, GLOVSKY, AND POPEO, P.C., ONE FINANCIAL CENTER, BOSTON, MA, 02111, US

(21) Appl. No.: 10/434,603

(22) Filed: May 9, 2003

Related U.S. Application Data
(63) Continuation of application No. PCT/US01/47306, filed on Nov. 9, 2001.
(60) Provisional application No. 60/247,592, filed on Nov. 9, 2000.
(60) Provisional application No. 60/250,728, filed on Dec. 1, 2000.

(51) Int. Cl. A61K 48/00; A61K 39/00

(52) U.S. Cl. 424/277.1; 424/93.21

(57) ABSTRACT

The present invention relates to autologous tumor vaccines that include tumor cells which have been genetically engineered to secrete SDF-1β. Also featured are methods of making such tumor vaccines as well as methods for vaccinating and treating subjects having cancer with the vaccines of the present invention.

### RELATED APPLICATIONS

This application claims priority to U.S. Provisional Application No. 60/247,592 entitled “SDF-1β Tumor Vaccines and Uses Therefor” filed on Nov. 9, 2000 and No. 60/250,728 entitled “SDF-1β Tumor Vaccines and Uses Therefor” filed Dec. 1, 2000, the entire contents of which are incorporated herein by reference.

### BACKGROUND OF THE INVENTION

Stromal cell-derived factor-1 (SDF-1) is a member of the CXC family of chemokines that is essential for perinatal viability, B lymphopoiesis, and bone marrow myelopoiesis, and also acts as both a highly efficacious and highly potent chemoattractant for T cells, lymphocytes, monocytes and hematopoietic progenitor cells. SDF-1 is also known to be a potent pre-B cell growth stimulating factor and has been reported to act together with interleukin-7 as a co-mitogen for pre-B cells. D'Apuzzo et al. (1997) Eur. J. Immunol. 27:1788-1793. Moreover, recent reports have demonstrated that SDF-1 has a growth promoting activity on human peripheral T cells, and it has been suggested that this activity as well as T cell migration, potentiation and maintenance may proceed via a mechanism involving activation of the MAP kinase, ERK2. Yonezawa et al. (2000) Microbiol. Immunol. 44:135-141.

The receptor for SDF-1, CXCR4, is broadly expressed in cells of both the immune system and the central nervous system and has recently been shown to be involved in the entry of T-tropic human immunodeficiency virus (HIV) into target CD4⁺ T cells. SDF-1, by downregulating cell surface CXCR4, is cabable of inhibiting HIV infection of cells expressing the receptor. Signoret et al. (1997) J. Cell. Biol. 139:651-64. CXCR4, is also expressed on endothelial cells and has been proposed to play a role in promoting angiogenesis both in vitro and in vivo.

Given the important role of SDF-1 and the SDF-1 receptor, CXCR4, in regulating diverse physiological activities ranging from hematopoiesis to HIV infection, this chemokine and its receptor have been proposed as potential targets for therapeutic intervention in neurodegenerative diseases, as well as in the development of new therapeutic agents for HIV infection and other immune system diseases.

### SUMMARY OF THE INVENTION

The present invention features a heretofore undescribed therapeutic use for SDF-1 which is based at least in part, on the discovery of a novel antitumor activity of SDF-1β. In particular, the present inventors have demonstrated that human SDF1β (hSDF-1β) secreted at a tumor site in an animal by genetically modified tumor cells initiates local immune responses that lead to tumor rejection and development of antitumor memory responses. In various animal tumor models, 40%-100% of the animals injected with hSDF-1β expressing tumor cells rejected their tumors. Moreover, animals developed long-lasting memory T cells, were immune to tumor rechallange and exhibited tumor specific CTL activity.

Based on this previously unrecognized antitumor activity of SDF-1β, the present invention features a vaccine comprising tumor cells isolated from a subject which have been modified to secrete an increased level of SDF-1β relative to unmodified tumor cells, wherein said vaccine confers tumor immunity upon administration to said subject.

In one embodiment, modifying said tumor cells comprises transducing said cells with a nucleic acid molecule which encodes SDF-1β.

In another embodiment, the nucleic acid molecule which encodes SDF-1β is in the form of a vector.

In another embodiment, the vector is a recombinant expression vector.

In still another embodiment, the recombinant expression vector is selected a viral expression vector.

In another embodiment, the recombinant expression vector is a replication-defective retroviral vector.

In another embodiment, the modified tumor cells have been expanded in culture prior to introduction of said nucleic acid molecule which expresses SDF-1β.

In one embodiment, the vaccine further comprises a pharmaceutically acceptable carrier.

In another aspect, the invention pertains to a method for producing an autologous tumor vaccine comprising:

(a) isolating tumor cells from a subject having cancer; and

(b) modifying said tumor cells such that they secrete an increased level of SDF-1β relative to unmodified tumor cells; such that an autologous tumor vaccine is produced.

In one embodiment, the cells to be modified are isolated from a tumor which has been surgically removed from said subject.

In another embodiment, the cells to be modified are isolated from a biopsy of a tumor in said subject.

In another embodiment, the cells to be modified are expanded in culture prior to modification of said cells.

In still another aspect, the invention pertains to a method for treating a subject having cancer comprising administering to said subject the autologous tumor vaccine of claim 1 in an amount sufficient to inhibit tumor growth, such that said subject is treated.

In one embodiment, the autologous tumor vaccine is administered when the tumor burden of said subject is low.

In another embodiment, the autologous tumor vaccine is administered after said subject has undergone chemotherapy.

In yet another embodiment, the autologous tumor vaccine is administered after said subject has undergone radiation therapy.

In another embodiment, the method further comprises monitoring the antitumor immune response in said subject

In one embodiment, the cells of said tumor vaccine are irradiated prior to administration to said subject.

In another embodiment, the cells of said tumor vaccine are admixed with an adjuvant prior to administration.

In yet another embodiment, the tumor vaccine is administered at or near at least one site of a tumor in said subject.

In yet another embodiment, the tumor vaccine is administered at or near at least one site from which a tumor has been surgically removed from said subject.

In still another aspect, the invention pertains to a method for promoting an antitumor response in a subject having cancer comprising administering to said subject the autologous tumor vaccine of claim 1, such that said subject develops an antitumor response to said vaccine.

In another embodiment, the autologous tumor vaccine is administered when the tumor burden of said subject is low.

In still another embodiment, the autologous tumor vaccine is administered after said subject has undergone chemotherapy.

In one embodiment, the autologous tumor vaccine is administered after said subject has undergone radiation therapy.

In another embodiment, the method further comprises monitoring the antitumor immune response in said subject

In another embodiment, the cells of said tumor vaccine are irradiated prior to administration to said subject.

In yet another embodiment, the cells of said tumor vaccine are admixed with an adjuvant prior to administration.

In still another embodiment, the tumor vaccine is administered at or near at least one site of a tumor in said subject.

In yet another embodiment, tumor vaccine is administered at or near at least one site from which a tumor has been surgically removed from said subject.

### BRIEF DESCRIPTION OF THE DRAWINGS

FIG. 1a shows ELISA results depicting the expression of SDF-1β by tumor cells

FIG. 1b depicts the comparison in morphology of cultured wild type (left panel) and SDF-1β-MB49 (right panel) bladder carcinoma cells.

FIG. 2 shows a survival curve depicting the tumorigenicity of SDF-1β-C1498 tumor cells.

FIG. 3a shows a survival curve depicting the tumorigenicity of SDF-1β-C1498 tumor cells.

FIG. 3b shows a survival curve depicting the tumorigenicity of SDF-1β-AML tumor cells.

FIG. 3c shows a survival surve depicting the tumorigenicity of SDF-1β-B16F1 tumor cells.

FIG. 3d shows a survival curve depicting the tumorigenicity of SDF-1β-MB49 tumor cells.

FIG. 4 shows a survival curve depicting the tumorigenicity of SDF-1β-AML and SDF-1β-TSA tumor cells.

FIG. 5 shows a survival curve depicting the tumorigenicity SDF-1β-MB49 tumor cells and gross analysis of SDF-1β-MB49 induced tumor masses.

FIGS. 6a and 6b depict survival curves demonstrating that irradiated SDF-1β-tumor cells support the induction of systemic prophylactic and therapeutic immunity.

FIG. 7a depicts a survival curve demonstratng that SDF-1β-tumor rejection supports the development of antitumor memory T cells.

FIG. 7b depicts a graph showing ⁵¹Cr release CTL assays of splenocytes isolated from naive mice and SDF-1β-B16F1 tumor-bearing mice.

FIG. 7c depicts a survival curve showing that CD4⁺ T cells are indespensible for SDF-1β-mediated tumor rejection.

FIGS. 8a and 8b depicts a survival curve demonstrating that scid mice do not reject SDF tumors.

FIG. 9 shows immunohistochemical data demonstrating that T cells infiltrate SDF-1β-B16F1, but not wild-type B16F1 tumors.

FIG. 10 shows flow cytomtery data demonstrating that SDF-1β-B16F1 cells restore CXCR4 expression on murine splenocytes.

FIG. 11 shows ³H-thymidine incorporation data demonstrating that SDF-1β-tumor cells significantly enhance in vitro proliferation of synergistic T cells.

### DETAILED DESCRIPTION OF THE INVENTION

The present invention is based at least in part, on the discovery of a novel antitumor activity of SDF-1β also referred to herein as SDF, SDF-1β and hSDF-1β). In particular, the present inventors have shown that human SDF-1β (hSDF-1β) secreted at a tumor site in an animal by genetically modified tumor cells initiates local immune responses that lead to tumor rejection and development of antitumor memory responses. This novel activity has been demonstrated in a variety of animal tumor models including radiation-induced acute myeloid leukemia (AML), C1498 leukemia, B16F1 melanoma and MB49 bladder carcinoma. The expression of SDF-1β by modified tumor cells induces morphological and phenotypical changes of transduced tumor cells but does not have an effect on the in vitro growth characteristics of the modified cells. In all tumor models tested, 40%-100% of the animals injected with hSDF-1β expressing tumor cells rejected their tumors. Animals that had previously rejected live SDF-1β-expressing-tumor cells developed long-lasting memory T cells, were immune to rechallange with live wild-type tumor cells, and exhibited tumor specific CTL activity. Animals that has previously been immunized with irradiated SDF-1β transduced tumor cells at one site were protected against inoculation at a second site with live wild-type tumor cells. Finally, tumor cells engineered to secrete increased levels of SDF-1β were not rejected by immunodeficeint animals and histological analysis showed heavy cellular infiltrates with immune cells surrounding tumor masses that secrete SDF-1β. These data collectively demonstrate a previously unrecognized antitumor activity of SDF-1β leading to the development of new and promising therapeutic approaches in the treatment of cancer.

Accordingly, a first aspect the present invention features autologous tumor vaccines. In one embodiment, an autologous tumor vaccine is featured that includes tumor cells from a subject (e.g., a subject or patient having cancer) the tumor cells having been modified to secrete an increased level of SDF-1β in comparison to the amount of SDF-1β secreted by unmodified tumor cells. In another embodiment, an autologous tumor vaccine is featured that includes tumor cells from a subject which have been modified to secrete an increased level of SDF-1β in comparison to the amount of SDF-1β secreted by unmodified tumor cells, the vaccine conferring tumor immunity following administration to the subject. In another embodiment, an autologous tumor vaccine is featured that includes tumor cells from a subject which have been modified to secrete an increased level of SDF-1β in comparison to the amount of SDF-1β secreted by unmodified tumor cells, the vaccine conferring tumor immunity following administration to the subject during a period of low tumor burden.

In one embodiment, the tumor cells are modified to secrete an increased level of SDF1β by introducing into the cells a nucleic acid molecule which encodes SDF-1β. An exemplary nucleic acid molecule is the nucleic acid molecule set forth as SEQ ID NO:1 (in particular, for use in human autologous tumor vaccines), which encodes a human SDF-1β protein having the amino acid sequence as set forth in SEQ ID NO:2. Additional exemplary nucleic acid molecules include those encoding variants (e.g., functional variants) of the human SDF-1β protein set forth as SEQ ID NO:2. (e.g., nucleic acid molecules having at least 90% identity to the nucleic acid molecule having the nucleotide sequence set forth as SEQ ID NO:1 and/or nucleic acid molecules which encode SDF-1β proteins having at least 90% identity to the polypeptide set forth as SEQ ID NO:2). Additional exemplary nucleic acid molecules include those encoding variants (e.g., functional variants) of the protein set forth as SEQ ID NO:2, incuding those which hybridize under stringent hybridization conditions to the nucleic acid molecule having the nucleotide sequence of SEQ ID NO:1.

The nucleic acid molecule which encodes SDF-1β can, for example, be introduced in the form of a vector (e.g., a secreted retroviral vector). A preferred recombinant expression vector is a replication-defective retroviral vector. In another embodiment of the invention, the modified tumor cells of the vaccine are expanded in culture prior to introduction of the nucleic acid molecule which expresses SDF-1β. Other embodiments of the present invention include tumor vaccines that include modified tumor cells in addition to a pharmaceutically acceptable carrier and/or adjuvant to further enhance the subjects immune response to the vaccine.

Another aspect of the present invention features methods for producing autologous tumor vaccines. In particular, a method is featured which includes isolating tumor cells from a subject or patient (e.g., a subject or patient having cancer) and modifying the isolated tumor cells such that they secrete an increased level of SDF-1β as compared to unmodified tumor cells, such that an autologous tumor vaccine is produced. In one embodiment, the method includes modifying cells which have been isolated from a tumor which has been surgically removed from the subject or patient. In another embodiment, the method includes modifying cells which have been isolated from a biopsy of a tumor from the subject or patient. In yet another embodiment, the method includes expanding the isolated cells in culture prior to modification of the cells.

Another aspect of the present invention features methods for treating subjects or patients (e.g., subjects or patients having cancer). In particular, a method is featured which includes administering to the subject or patient an autologous tumor vaccine as defined herein and monitoring tumor growth and/or tumor regression in the subject (e.g., after administering the vaccine), such that the subject or patient is treated. In one embodiment the method also includes the step of determining that the subject or patient develops an immune response to said cells. In another embodiment, the method involves administering the vaccine when the subject or patient's tumor burden is low, e.g., when the tumor is detected at an early stage or after the subject has been treated using using another method (such as chemotherapy or radiation).

Yet another aspect of the present invention features methods for stimulating an antitumor response in a subject or patient having cancer. In particular, the invention features a method which involves administering to the subject or patient an autologous tumor vaccine as defined herein, such that the subject or patient develops an antitumor response to the vaccine. In certian embodiments, the methods for treating or stimulating an antitumor response in a subject or patient having cancer involve administering the autologous tumor vaccine after the subject or patient has undergone chemotherapy. In other embodiments, the methods involve administering the autologous tumor vaccine after the subject has undergone radiation therapy. In yet other embodiments, the methods involve irradiating the cells of the autologous tumor vaccine prior to administration to the subject or patient and/or admixing the cells with an adjuvant prior to administration. In yet other embodiments, the methods invlove administering the tumor vaccine at or near a tumor site in the subject or patient or at or near a site from which a tumor has been surgically removed from the subject or patient. In yet another embodiment, the method of stimulating an antitumor response also involves monitoring the antitumor response in said subject

In order that the present invention may be more readily understood, certain terms are first defined herein.

As used herein, the term “immune cell” includes cells that are of hematopoietic origin and that play a role in the immune response. Immune cells include lymphocytes, such as B cells and T cells; natural killer cells; myeloid cells, such as monocytes, macrophages, eosinophils, mast cells, basophils, and granulocytes.

As used herein, the term “T cell” includes CD4+ T cells and CD8+ T cells. The term T cell also includes both T helper 1 type T cells and T helper 2 type T cells. The term “antigen presenting cell” includes professional antigen presenting cells (e.g., B lymphocytes, monocytes, dendritic cells, Langerhans cells) as well as other antigen presenting cells (e.g., keratinocytes, endothelial cells, astrocytes, fibroblasts, oligodendrocytes).

As used herein, the term “immune response” includes T cell mediated and/or B cell mediated immune responses. Exemplary immune responses include T cell responses, e.g., cytokine production, and cellular cytotoxicity. In addition, the term immune response includes immune responses that are indirectly effected by T cell activation, e.g., antibody production (humoral responses) and activation of cytokine responsive cells, e.g., macrophages.

The term “vaccine” as used herein, includes a composition (e.g., a suspension) of antigens or cells, preferably attenuated cells or organisms, which produces or elicits an immune response (e.g., produces or elicits active immunity) when administered to a subject.

As used herein, the term “tumor” includes a neoplastic growth, either benign or malignant. The term “tumor vaccine” includes a vaccine containing tumor cells or tumor cell antigens capable of producing or eliciting an immune response. The term “tumor cell” includes a cell, either derived from or forming the source of a tumor, such cell characterized by excessive, abnormal, deregulated or uncontrolled proliferation or multiplication. Preferred tumor cells are those of epithelial or hematopoetic origin.

The term “autologous” means produced by or derived from the body of the subject in question (e.g., produced by or derived from the body of a subject being administered a vaccine or being treated), for example, an autologous protein, cell or tissue (e.g. an autologous tissue sample or graft). The phrase “autologous tumor vaccine” includes a tumor vaccine, as defined herein, wherein the tumor cells or tumor cell antigens of the vaccine are produced by or derived from the body of the subject being administered the vaccine or treated according to at least one of the therapeutic methodologies described herein.

The term “antitumor response” includes an immune response to a tumor, tumor cells, or any portion of said tumor cells, for example a response to tumor antigens present on the surface of the tumor cells.

The term “cancer” includes malignant neoplastic growths, in particular those of epithelial or hematopoietic origin, characterized by abnormal cellular proliferation and the absence of contact inhibition. The term encompasses cancer localized in tumors, as well as cancer not localized in tumors, such as, for instance, cancer cells which expand from a tumor locally by invasion. Thus, any type of cancer can be targeted for treatment according to the invention. For example, the methodologies described herein preferably can be applied in several clinical scenarios including, but not limited to, local adjuvant therapy for resected cancers, and local control of tumor growth, such as carcinomas of the bladder, breast, colon, kidney, liver, lung, ovary, pancreas, rectum, and stomach. The method also preferably can be used for treatment when the tumor is a sarcoma (e.g., a fibrosarcoma or rhabdosarcoma), a hematopoietic tumor of lymphoid or myeloid lineage, or another tumor, including, but not limited to, a melanoma, teratocarcinoma, neuroblastoma, or glioma.

The term “subject” includes various living mammalian subjects including but not limited to rodents, primates, domestic mammals (such as feline and canine), farm animals (such as ruminant or swine), and in particular, human subjects. Accordingly, the phrase “subject having cancer” includes, any subject, including but not limited to the aforementioned subjects, exibiting the symptoms of cancer, having been diagnosed with cancer, or having as yet undiagnosed uncontrolled cellular proliferation, polyp(s), tumor(s), or any other phenotypic manifestation of cancer as defined herein. In a preferred embodiment, a subject having cancer is a human subject. In another preferred embodiment, a subject having cancer is a human patient (i.e., a human subject having been diagnosed as having cancer and/or under the care of a health care profesional for the treatment of cancer).

“Treating cancer” according to the invention comprises administering to a subject having cancer a compound, agent, pharmaceutical or treatment, preferably an autologous tumor vaccine of the present invention, for the purpose of effecting a therapeutic response. Preferably the response can be assessed by monitoring the attenuation of tumor growth and/or tumor regression. “Tumor growth” includes an increase in tumor size and/or the number of tumor cells or in the number of tumors. “Tumor regression” includes a reduction in tumor mass.

Various aspects of the invention are described in further detail in the following subsections.

I. Ex Vivo Modification Tumor Cells to Express SDF-1β

The present invention features autologous tumor vaccines that include cells which have been modified or engineered to express the chemokine SDF-1β at a level greater than that expressed prior to modification or in a comparable unmodified tumor cell or tumor cell population. Tumor cells suitable for use in the preparation of the vaccines of the present invention can be isolated from solid tumors present in a subject having cancer or can be isolated from biological fluids of a patient having a cancer that is hematopoeitic in nature. The tumor cells can be obtained, for example, from a solid tumor of an organ, including but not limited to a carcinoma of the bladder, breast, colon, kidney, liver, lung, ovary, pancreas, rectum, or stomach; or can be obtained from a hematopoietic tumor of lymphoid or myeloid lineage (e.g., a lymphoma, myeloma or leukemia); a tumor of mesenchymal origin such as a fibrosarcoma or rhabdomyosarcoma; or another tumor, including a melanoma, teratocarcinoma, neuroblastoma, or glioma. Preferably the tumor cell is derived from a tumor of epithelial or hematopoeitic origin.

Such tumor cells can be isolated by any suitable means but preferably is isolated in a general method involving the steps of (a) obtaining a sample of a tumor from a subject (e.g., a human subject), (b) harvesting tumor cells from the tumor obtained, (c) forming a suspension of tumor cells (e.g., a single cell suspension), and (d) culturing the tumor cells.

For example, tumor cells can be obtained from a subject by, for example, surgical removal of tumor cells, e.g. a biopsy of the tumor, or from a blood sample from the subject in cases of blood-borne malignancies. In the case of an experimentally induced tumor, the cells used to induce the tumor can be used, e.g. cells of a tumor cell line. Samples of solid tumors may be treated prior to modification to produce a single-cell suspension of tumor cells for maximal efficiency of transfection. Possible treatments include manual dispersion of cells or enzymatic digestion of connective tissue fibers, e.g. by collagenase.

Tumor cells can be transfected immediately after being obtained from the subject or can be cultured in vitro prior to transfection to allow for further characterization of the tumor cells (e.g. determination of the expression of cell surface molecules).

Prior to administration to the subject, the modified tumor cells can be treated to render them incapable of further proliferation in the subject, thereby preventing any possible outgrowth of the modified tumor cells. Possible treatments include irradiation or mitomycin C treatment, which abrogate the proliferative capacity of the tumor cells while maintaining the ability of the tumor cells to stimulate T cells and thus to stimulate an immune response.

More specifically, a sample of a tumor typically can be obtained at the time of surgery. The tumor sample subsequently can be handled and manipulated using sterile technique and in such a fashion so as to minimize tissue damage. The tissue sample can be placed on ice in a sterile container and moved to a laboratory laminar flow hood. The portion of the tumor to be employed for isolation of tumor cells can be minced into small pieces; the remainder of the tumor can be stored at −70° C. The pieces of tumor then can be digested into single cell suspensions using a solution of collagenase, trypsin, or another suitable digestive enzyme. This digestion can be carried out at room or at elevated temperature. Preferably the digestion is carried out at 37° C. while shaking the mixture. e.g., in a shaking incubator.

The single cell suspension is then pelleted, and the pellets can be resuspended in a small volume of tissue culture medium. The resuspended cells can be inoculated into tissue culture medium appropriate for the growth of the cells in culture at a density of about 1-5×10⁵ tumor cells/ml. Preferably the medium is one that has wide applicability for supporting growth of many types of cell cultures, such as a medium that utilizes a bicarbonate buffering system and various amino acids and vitamins. Optimally the medium is RPMI-1640 medium. The medium can contain various additional factors as necessary, e.g., when required for the growth of tumor cells or for maintenance of the tumor cells in an undifferentiated state.

The cultures can be maintained at about 35-40° C., in the presence of about 5-7% CO₂. The tumor cell cultures can be fed and recultured as necessary. As part of the isolation process, the tumor cells can be plated in a growth medium optimized for culturing tumor cells. Preferably, this medium further comprises serum (e.g., fetal serum) and/or growth factors, for example insulin and/or insulin-like growth factors. The medium and medium components are readily available, and can be obtained, for instance, from commercial suppliers. Such commercial suppliers include, but are not limited to Gibco BRL (Gaithersberg, Md.), Hyclone Laboratories (Logan, Utah), Sigma Biosciences (St. Louis, Mo.) and other suppliers manufacturing similar products.

Unmodified tumor cells do not produce detecatble levels of SDF-1β. Accordingly, it is necessary to modify the tumor cells isolated as described above in order that SDF-1β is produced. As used herein, the term “modified” or “modification” included engineering or manipulating the cell such that expression of SDF-1β nucleic acid molecules, expression or production of SDF-1β polypeptides and/or secretion of SDF-1β is increased to a level greater than that expressed, produced or secreted prior to engineering or manipulation of the cell or in a comparable cell which has not been engineered or manipulated. Genetic manipulation can include, but is not limited to, transfection of the tumor cell or tumor cell population with nucleic acid seqeunces which encode SDF-1β. The terms “transfection” or “transfected with” refers to the introduction of exogenous nucleic acid into a cell (e.g., a mammalian cell) and encompass a variety of techniques useful for introduction of nucleic acids into mammalian cells including electroporation, calcium-phosphate precipitation, DEAE-dextran treatment, lipofection, microinjection and infection with viral vectors. Suitable methods for transfecting mammalian cells can be found in Sambrook et al. (Molecular Cloning: A Laboratory Manual 2nd Edition, Cold Spring Harbor Laboratory press (1989)) and other laboratory textbooks. The nucleic acid to be introduced can be, for example, DNA encompassing the gene encoding SDF-1β, sense strand RNA encoding the SDF-1β or a recombinant expression vector containing a cDNA encoding SDF-1β. If necessary, following modification, tumor cells can be screened for introduction of the nucleic acid by using a selectable marker (e.g. drug resistance) which is introduced into the tumor cells together with the nucleic acid of interest.

A preferred approach for introducing SDF-1β-encoding nucleic acid sequences into tumor cells is by use of a viral vector containing the nucleic acid sequences, e.g. a cDNA, encoding SDF-1β. Examples of viral vectors which can be used include retroviral vectors (Eglitis, M. A., et al., Science 230, 1395-1398 (1985); Danos, O. and Mulligan, R., Proc. Natl. Acad. Sci. USA 85, 6460-6464 (1988); Markowitz, D., et al., J. Virol. 62, 1120-1124 (1988)), adenoviral vectors (Rosenfeld, M. A., et al., Cell 68, 143-155 (1992)) and adeno-associated viral vectors (Tratschin, J. D., et al., Mol. Cell. Biol. 5, 3251-3260 (1985)). Infection of tumor cells with a viral vector has the advantage that a large proportion of cells will receive nucleic acid, thereby obviating a need for selection of cells which have received nucleic acid, and molecules encoded within the viral vector, e.g. by a cDNA contained in the viral vector, are expressed efficiently in cells which have taken up viral vector nucleic acid.

According to the invention, increasing the level or amount of SDF-1β secreted by a tumor cell can be accomplished, at least in one embodiment, introducing into cells a nucleic acid molecule which encodes SDF-1β. The term “nucleic acid molecule”, as used herein, includes DNA molecules (e.g., linear, circular or chromosomal DNA molecules) and RNA molecules (e.g., tRNA, rRNA, mRNA) and analogs of the DNA or RNA generated using nucleotide analogs. The nucleic acid molecule can be single-stranded or double-stranded, but preferably is double-stranded DNA The term “isolated” nucleic acid molecule includes a nucleic acid molecule which is free of sequences which naturally flank the nucleic acid molecule, for example, contains less than about 10 kb, 5 kb, 2 kb, 1 kb, 0.5 kb, 0.2 kb, 0.1 kb, 50 bp, 25 bp or 10 bp of nucleotide sequences which naturally flank the naturally occurring nucleic acid sequence in the organism from which the nucleic acid molecule is derived. Preferably, an “isolated” nucleic acid molecule, such as a DNA molecule, is substantially free of other cellular materials when produced by recombinant techniques, or substantially free of chemical precursors or other chemicals when chemically synthesized.

An exemplary nucleic acid molecule is the nucleic acid molecule set forth as SEQ ID NO:1 (in particular, for use in human autologous tumor vaccines), which encodes a human SDF-1β protein having the amino acid sequence as set forth in SEQ ID NO:2. Additional exemplary nucleic acid molecules include those encoding functional variants of the human SDF-1β protein set forth as SEQ ID NO:2. As used herein, a functional variant of the SDF-1β protein set forth as SEQ ID NO:2 includes a protein having at least 90%, preferably at least 95%, 96%, 97%, 98%, 99%, or more identity to the human SDF-1β sequence set forth as SEQ ID NO:2 and sharing substantially the same biological activity as the human SDF-1β sequence set forth as SEQ ID NO:2, including but not limited to nucleic acid molecules having at least 90%, preferably at least 95%, 96%, 97%, 98%, 99%, or more identity to the nucleic acid molecule having the nucleotide sequence set forth as SEQ ID NO:1. For example, variants having conserved substitutions at various positions within the amino acid sequence (or having non-conserved substitutions, or minimal insertions of deletions, for example, at non-essential residue positions) can retain the biological activity of the human SDF-1β sequence.

To determine the percent homology of two amino acid sequences or of two nucleic acids, the sequences are aligned for optimal comparison purposes (e.g., gaps and/or insertions can be introduced in the sequence of a first amino acid or nucleic acid sequence for optimal alignment with a second amino or nucleic acid sequence). When a position in the first sequence is occupied by the same amino acid residue or nucleotide as the corresponding position in the second sequence, then the molecules are said to be identical at that position. The percent identity between the two sequences is a function of the number of identical positions shared by the sequences (i.e., % identity=# of identical positions/total # of positions×100), preferably taking into account the number of gaps and size of said gaps necessary to produce an optimal alignment.

The comparison of sequences and determination of percent homology between two sequences can be accomplished using a mathematical algorithm. A preferred, non-limiting example of a mathematical algorithm utilized for the comparison of sequences is the algorithm of Karlin and Altschul (1990) Proc. Natl. Acad. Sci. USA 87:2264-68, modified as in Karlin and Altschul (1993) Proc. Natl. Acad. Sci. USA 90:5873-77. Such an algorithm is incorporated into the NBLAST and XBLAST programs (version 2.0) of Altschul et al. (1990) J. Mol. Biol. 215:403-10. BLAST nucleotide searches can be performed with the NBLAST program, score=100, wordlength=12 to obtain nucleotide sequences homologous to nucleic acid molecules of the invention. BLAST protein searches can be performed with the XBLAST program, score=50, wordlength=3 to obtain amino acid sequences homologous to protein molecules of the invention. To obtain gapped alignments for comparison purposes, Gapped BLAST can be utilized as described in Altschul et al. (1997) Nucleic Acids Research 25(17):3389-3402. When utilizing BLAST and Gapped BLAST programs, the default parameters of the respective programs (e.g., XBLAST and NBLAST) can be used. See http://www.ncbi.nlm.nih.gov. Another preferred, non-limiting example of a mathematical algorithm utilized for the comparison of sequences is the algorithm of Myers and Miller (1988) Comput Appl Biosci. 4:11-17. Such an algorithm is incorporated into the ALIGN program available for example, at the GENESTREAM network server, IGH Montpellier, FRANCE or at the ISREC server. When utilizing the ALIGN program for comparing amino acid sequences, a PAM120 weight residue table, a gap length penalty of 12, and a gap penalty of 4 can be used.

In another preferred embodiment, the percent homology between two amino acid sequences can be accomplished using the GAP program in the GCG software package (Washington University web server), using either a Blossom 62 matrix or a PAM250 matrix, and a gap weight of 12, 10, 8, 6, or 4 and a length weight of 2, 3, or 4. In yet another preferred embodiment, the percent homology between two nucleic acid sequences can be accomplished using the GAP program in the GCG software package, using a gap weight of 50 and a length weight of 3.

Additional exemplary nucleic acid molecules include those encoding varaints of the protein set forth as SEQ ID NO:2, incuding those which hybridize under stringent hybridization conditions to the nucleic acid molecules having the nucleotide sequence of SEQ ID NO:1. In one embodiment, an isolated SDF1β encoding nucleic acid molecule hybridizes under stringent conditions to all or a portion of the nucleic acid molecule having the nucleotide sequence set forth in SEQ ID NO:1 or hybridizes to all or a portion of a nucleic acid molecule having a nucleotide sequence that encodes the polypeptide having the amino acid sequence set forth as SEQ ID NO:2 Such stringent conditions are known to those skilled in the art and can be found in Current Protocols in Molecular Biology, Ausubel et al., eds., John Wiley & Sons, Inc. (1995), sections 2, 4 and 6. Additional stringent conditions can be found in Molecular Cloning: A Laboratory Manual, Sambrook et al., Cold Spring Harbor Press, Cold Spring Harbor, N.Y. (1989), chapters 7, 9 and 11. A preferred, non-limiting example of stringent hybridization conditions includes hybridization in 4×sodium chloride/sodium citrate (SSC), at about 65-70° C. (or hybridization in 4×SSC plus 50% formamide at about 42-50° C.) followed by one or more washes in 1×SSC, at about 65-70° C. A preferred, non-limiting example of highly stringent hybridization conditions includes hybridization in 1×SSC, at about 65-70° C. (or hybridization in 1×SSC plus 50% formamide at about 42-50° C.) followed by one or more washes in 0.3×SSC, at about 65-70° C. A preferred, non-limiting example of reduced stringency hybridization conditions includes hybridization in 4×SSC, at about 50-60° C. (or alternatively hybridization in 6×SSC plus 50% formamide at about 40-45° C.) followed by one or more washes in 2×SSC, at about 50-60° C. Ranges intermediate to the above-recited values, e.g., at 65-70° C. or at 42-50° C. are also intended to be encompassed by the present invention. SSPE (1×SSPE is 0.15 M NaCl, 10 mM NaH₂PO₄, and 1.25 mM EDTA, pH 7.4) can be substituted for SSC (1×SSC is 0.15 M NaCl and 15 mM sodium citrate) in the hybridization and wash buffers; washes are performed for 15 minutes each after hybridization is complete. The hybridization temperature for hybrids anticipated to be less than 50 base pairs in length should be 5-10° C. less than the melting temperature (Tₘ) of the hybrid, where Tₘ is determined according to the following equations. For hybrids less than 18 base pairs in length, Tₘ(° C.)=2(# of A+T bases)+4(# of G+C bases). For hybrids between 18 and 49 base pairs in length, Tₘ(° C.)=81.5+16.6(log₁₀[Na⁺])+0.41(% G+C)−(600/N), where N is the number of bases in the hybrid, and [Na⁺] is the concentration of sodium ions in the hybridization buffer ([Na⁺] for 1×SSC=0.165 M). It will also be recognized by the skilled practitioner that additional reagents may be added to hybridization and/or wash buffers to decrease non-specific hybridization of nucleic acid molecules to membranes, for example, nitrocellulose or nylon membranes, including but not limited to blocking agents (e.g., BSA or salmon or herring sperm carrier DNA), detergents (e.g., SDS), chelating agents (e.g., EDTA), Ficoll, PVP and the like. When using nylon membranes, in particular, an additional preferred, non-limiting example of stringent hybridization conditions is hybridization in 0.25-0.5M NaH₂PO₄, 7% SDS at about 65° C., followed by one or more washes at 0.02M NaH₂PO₄, 1% SDS at 65° C., see e.g., Church and Gilbert (1984) Proc. Natl. Acad. Sci. USA 81:1991-1995, (or, alternatively, 0.2×SSC, 1% SDS). In another preferred embodiment, an isolated nucleic acid molecule comprises a nucleotide sequence that is complementary to an SDF-1β-encoding nucleotide sequence as set forth herein (e.g., is the full complement of the nucleotide sequence set forth as SEQ ID NO:1).

Additional SDF-1β-encoding nucleic acid molecules (e.g., encoding additional SDF-1β varaints) can be isolated using standard molecular biology techniques and the sequence information provided herein. For example, nucleic acid molecules can be isolated using standard hybridization and cloning techniques (e.g., as described in Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, N.Y., 1989) or can be isolated by the polymerase chain reaction using synthetic oligonucleotide primers designed based upon the SDF-1β-encoding nucleotide sequences set forth herein, or flanking sequences thereof. A nucleic acid of the invention can be amplified using cDNA, mRNA or alternatively, chromosomal DNA, as a template and appropriate oligonucleotide primers according to standard PCR amplification techniques.

Preferably, the SDF-1β-encoding nucleic acid seqeunces are “in a form suitable for expression” in which the nucleic acid contains all of the coding and regulatory sequences required for transcription and translation of the gene, which may include promoters, enhancers and polyadenylation signals, and sequences necessary for secretion of the molecule from the tumor cell, including N-terminal signal sequences. When the nucleic acid is a cDNA in a recombinant expression vector, the regulatory functions responsible for transcription and/or translation of the cDNA are often provided by viral sequences. Examples of commonly used viral promoters include those derived from polyoma, Adenovirus 2, cytomegalovirus and Simian Virus 40, and retroviral LTRs. Regulatory sequences linked to the CDNA can be selected to provide constitutive or inducible transcription, by, for example, use of an inducible promoter, such as the metallothienin promoter or a glucocorticoid-responsive promoter. Secretion of SDF-1β from the tumor cell can be accomplished, for example, by including a native signal sequence of the molecule in the nucleic acid sequence, or by including signals which lead to increased secretion of the protein, such as a heterologous signal sequence.

In a preferred embodiment, SDF-1β is secreted from the modified tumor cell at a level greater than about 0.1 ng/10⁶ cell/24 hours, preferably greater than 1 ng/10⁶ cell/24 hours, more preferably greater than 10 ng/10⁶ cell/24 hours, more preferably greater than 20 ng/10⁶ cell/24 hours, even more preferably greater than 25 ng/10⁶ cell/24 hours, even more preferably greater than 30 ng/10⁶ cell/24 hours, and even more preferably greater than 35 ng/10⁶ cell/24 hours.

When transfection of tumor cells leads to modification of a large proportion of the tumor cells and secretion of significant levels of SDF-1β from the tumor cells, e.g. when using a viral expression vector, tumor cells may be used without further isolation or subcloning. Alternatively, a homogenous population of transfected tumor cells can be prepared by isolating a single transfected tumor cell by limiting dilution cloning followed by expansion of the single tumor cell into a clonal population of cells by standard techniques.

The tumor cells to be modified as described herein include tumor cells which have been infected, transfected or treated by one or more of the approaches encompassed by the present invention to express, produce and/or secrete increased levels of SDF-1β. If necessary, the tumor cell can be further be treated prior to administration to prevent cell replication, and possible tumor formation in vivo. Possible treatments include mitomycin C treatment or irradiation, which abbrogate the proliferative capacity of the tumor cells while maintaining the ability of the tumor cells to stimulate an immune response. For irradiation of tumor cells, the tumor cells typically are plated in a tissue culture plate and irradiated at room temperature using a ¹³⁷Cs source. Preferably, the cells are irradiated at a dose rate of from about 50 to about 200 rads/min, even more preferably, from about 120 to about 140 rads/min. Preferably, the cells are irradiated with a total dose sufficient to inhibit the majority of cells, i.e., preferably about 100% of the cells, from proliferating in vitro. Thus, desirably the cells are irradiated with a total dose of from about 10,000 to 20,000 rads, optimally, with about 15,000 rads.

Moreover, the modified tumor cells (e.g., the SDF-1β-expressing tumor cells) optionally are treated prior to administration to enhance the immunogenicity of the cells. Preferably, this treatment comprises admixture with nonspecific adjuvants including but not limited to Freund's complete or incomplete adjuvant, emulsions comprised of bacterial and mycobacterial cell wall components, and the like. Alternatively, further genetic manipulation, for example introduction of cytokine or immune co-stimulatory functions is intended to be within the scope of the present invention.

II. Administering Tumor Vaccines

“Administering” the tumor cell vaccines of the present invention to a subject refers to the actual physical introduction of the modified (i.e., SDF-1β-producing) tumor cells into the subject. Any and all methods of introducing the modified tumor cells into the subject are contemplated according to the invention; the method is not dependent on any particular means of introduction and is not to be so construed. Means of introduction are well known to those skilled in the art, and also are exemplified herein.

In one embodiment, the modified tumor cells are administered to the subject by injection of the tumor cells into the subject. The route of injection can be via any route that allows for optimal immune response by the subject to the tumor cells and may vary depending upon the type of tumor involved. For example, administration can be intravenous, intramuscular, intraperitoneal or subcutaneous. Administration of the modified tumor cells at the site of the original tumor (e.g., a resected tumor) may be beneficial for inducing local immune responses, e.g., T cell or B cell-mediated immune responses. Administration of the modified tumor cells in a disseminated manner, e.g. by intravenous injection, may provide systemic anti-tumor immunity and, furthermore, may protect against metastatic spread of tumor cells from the original site. Preferably the tumor cell vaccine (e.g., the SDF-1β-expressing tumor cell vaccine) is administered when the subjects tumor burden is low. This can be achieved, for example, by administration of the vaccine following irradiation, chemotherapy, surgical intervention (e.g., surgical resection), or the like. It is also within the scope of the invention to administer the modified tumor cells to a subject prior to or in conjunction with (i.e., concurrently or immediately following) other forms of therapy employed to treat cancer, for example, vaccination with cytokines, defined antigens (e.g., tumor antigens), idiotype antibody vaccination, and the like.

Another aspect of the invention is a composition of modified tumor cells in a biologically compatible form suitable for pharmaceutical administration to a subject in vivo. This composition comprises an amount of modified tumor cells and a physiologically acceptable carrier. The amount of modified tumor cells is selected to be therapeutically effective. The term “biologically compatible form suitable for pharmaceutical administration . . . in vivo” includes administration of any form of the vaccine in which toxic effects of the tumor cel vaccine is outweighed by the therapeutic effects of the tumor cells. A “physiologically acceptable carrier” is one which is biologically compatible with the subject. Examples of acceptable carriers include saline and aqueous buffer solutions. In all cases, the compositions must be sterile and preferably are fluid to the extent that easy syringability exists.

Administration of the therapeutic compositions of the present invention can be carried out using known procedures, at dosages and for periods of time effective to achieve the desired result. For example, a therapeutically effective dose of modified tumor cells may vary according to such factors as age, sex and weight of the individual, the type of tumor cell and degree of tumor burden, and the immunological competency of the subject. Dosage regimens may be adjusted to provide optimum therapeutic responses. For instance, a single dose of modified tumor cells may be administered or more than one dose may be administered over time.

Preferably, a sufficient number of the modified tumor cells are present in the pharmaceutical or therapeutic composition and introduced into the subject, such that a greater immune response to recurring tumors is present than would otherwise have been observed in the absence of such treatment, as further discussed herein. The dosage of modified tumor cells administered should take into account the route of administration and should be such that a sufficient number of the tumor cells will be introduced so as to achieve the desired therapeutic effect (e.g., tumor immunity). Furthermore, the amounts of each active agent included in the compositions described herein (e.g., the amount per each cell to be contacted or the amount per certain body weight) can vary in different applications. In an exemplary embodiment, the concentration of modified tumor cells can include greater than 0.5×10⁵ cells per ml of vaccine, preferably greater than 1×10⁵ cells per ml of vaccine, and optionally greater than 2×10⁵ cells or 1×10⁷ cells per ml of vaccine.

These values provide general guidance of the range of each component to be utilized by the practitioner upon optimizing the method of the present invention for practice of the invention. The recitation herein of such ranges by no means precludes the use of a higher or lower amount of a component, as might be warranted in a particular application. For example, the actual dose and schedule can vary depending on whether the compositions are administered in combination with other pharmaceutical compositions, or depending on interindividual differences in pharmacokinetics, drug disposition, and metabolism. One skilled in the art readily can make any necessary adjustments in accordance with the exigencies of the particular situation. Moreover, the effective amount of the compositions can be further approximated through analogy to other compounds known to inhibit the growth of cancer cells.

III. Therapeutic Methods Featuring SDF-1βProducing Tumor Cells

The therapeutic methods of the present invention can include, in addition to administering an autologous tumor vaccine, administration of one or more additional agents that further enhance the immune response against tumor cells in a subject. Such agents can be, e.g., coadministered with the vaccine or the tumor cells of a vaccine of the invention can be caused to coexpress such additional agents. For example, a tumor cell of the invention can be engineered to coexpress SDF-1 and another molecule, e.g., a costimualtory molecule (e.g., a B7-1 or B7-2 molecule) or other such immunostimulatory molecule (e.g., a cytokine), or a molecule which will cause the tumor cells of the vaccine to home to a particular site the the patient, e.g., a molecule that will cause the modified tumor cells of the vaccine to home to the site of the primary tumor (e.g., a homing receptor or an antibody specific for a specific cell type).

Preferably, the administration of a vaccine of the present invention to a subject results in one or more of the following: an increase in an immune response against the tumor cell in the subject, a decrease in angiogenesis in the subject (particularly at the site of the tumor), an increase in chemotaxis of immune cells of the subject to the site of the tumor, an increase in SDF receptor expression on cells of the subject, a decrease the recurrance of tumor regrowth in the subject, the promotion of tumor regression in the subject.

A variety of means can be used to monitor a therapeutic response upon administering a composition of the present invention. For example, in measuring an immune response to tumor cells one can measure, e.g., a specific antibody response to the tumor cells, B cell activation levels, T cell immune responses to tumor cells (e.g., CD4+ T cell responses(e.g., cytokine production or proliferation) or CD8+ T cell responses(e.g., cytotoxic T cell responses)), and/or chemotaxis of immune cells into the area of the tumor.

In one embodiment, the effect of a tumor cell vaccine of the invention on the immune response of the subject can be measured by assaying the immune response of the subject to, e.g., a specific tumor cell antigen or to tumor cells. This can be accomplished using standard techniques, e.g., by measuring the level of antibody in the subject that recognizes tumor cells or by measuring the ability of T cells obtained from the subject to respond to the tumor cells (e.g., by causing lysis of the tumor cells or by measuring proliferation of T cells from the subject in the presence of tumor cells in vitro).

In one embodiment, the primary response to a tumor cell can be measured, e.g., by measuring the immune response to tumor cells in a primary culture. In another embodiment, the secondary immune response to a tumor cell can be measured, e.g., by performing a primary culture in which immune cells of the subject are exposed to tumor cells, rested, and then reexposed to tumor cells.

Preferably, when measuring the immune response to a tumor cell in a subject treated using a modified tumor cell vaccine of the invention, unmodified tumor cells are used in performing the assays described herein.

In one embodiment, the levels of memory T cells in a subject treated using the subject vaccine can be measured, e.g., by assaying for cell surface markers preferentially expressed by memory T cells. In yet another embodiment, levels of tumor cell specific memory T cells can be assayed and preferably they are increased in a subject receiving a vaccine of the invention.

Additionally or alternatively, enhanced tumor regression or arrest or reduction of tumor cell growth can also be measured as an indication of an enhanced immune response to tumor cells.

In another embodiment, angiogenesis can be measured in the subject, to determine whether a decrease in angiogenesis, relative to an untreated subject (or to the subject prior to treatment) has occurred. Angiogenesis can be measured using techniques known in the art, e.g., by measuring for the presence of markers which are indicative of the presence of endothelial cells.

In another embodiment, an increase in the presence of T cells at the site of the primary tumor can be used as an indication of an enhanced immune response in the subject. SDF-1 is known to chemoattract T cells, and the modified tumor cells of the inventions produce SDF-1. In addition, although wild-type tumor cells (e.g., unmodified tumor cells) can downregulate SDF-1 receptor expression (at both the RNA and protein level), this downregulation does not occur in subjects receiving vaccines of SDF-1 producing cells. Therefore, in one embodiment, a subject receiving a vaccine of the present invention displays an increase in the level of chemokine receptors on cells (e.g., SDF-1 and/or Rantes) above that seen prior to treatment of the subject with the vaccine. Accordingly, enhanced presence of T cells at the site of a tumor in vivo can be used as an indication of an enhanced tumor cell response. The presence of T cells can be measured using any of a variety of techniques, e.g., T cells can be detected in a biopsy sample (e.g., by histology or FACS) by testing for T cell markers (e.g., CD3, CD4, or CD8). In another embodiment, levels of chemokine receptors, e.g., SDF-1 and Rantes) are enhanced on cells of a subject receiving a vaccine of the invention. The levels of such receptors can be tested using standard techniques, e.g., PCR, Northern blots, Western blots or FACS analysis.

In addition, the enhanced chemotaxis of T cells to tumor cells can be measured in vitro. This can be done using standard techniques, e.g., by showing enhanced chemotaxis of T cells obtained from the subject to tumor cells, e.g., on a slide or using a Boyden chamber.

In yet another embodiment, a therapeutic response obtained upon administration of a vaccine of the invention can be assessed by monitoring attenuation of tumor growth, and/or tumor regression. The attenuation of tumor growth or tumor regression in response to treatment with a modified tumor cell vaccine can be monitored using several end-points known to those skilled in the art including, for instance, number of tumors, tumor mass or size, or reduction/prevention of metastasis.

In addition or alternatively, the failure of the subject to relapse can be used to indicate that a vaccine of the invention has enhanced an immune response to a tumor cell. For examples, individuals with a particular type of cancer may suffer a relapse at a particular statistical rate. A decrease in, e.g., the rate of relapse or the amount of time to relapse can also be used as an indication that a subject has an enhanced immune response to a tumor cell.

The modified tumor cells of the current invention are also useful in a preventing or treating metastatic spread of a tumor or preventing or treating recurrence of a tumor, e.g., by inducing a memory response in subject. Accordingly, in one embodiment, an enhanced immune response induced by a tumor cell vaccine of the invention is evidenced by the ability of the subject to resist subsequent challenge with unmodified tumor cells (e.g., as evidenced by the greater resistance to the tumor challenge than an unvaccinated subject). Additionally or alternatvely, the ability of a subject treated with a tumor vaccine of the invention to mount a strong in vitro immune response (e.g., an anti-tumor cytotoxic response) against tumor cells can be used to demonstrate an enhanced anti-tumor response.

These described methods are by no means all-inclusive, and further methods to suit the specific application will be apparent to the ordinary skilled artisan.

This invention is further illustrated by the following examples which should not be construed as limiting. The contents of all references, patents and published patent applications cited throughout this application are incorporated herein by reference.

### EXAMPLES

The following examples further illustrate the present invention but, of course, should not be construed as in any way limiting its scope.

### Example I

Mice—Female SJL/J, C57BL/6 and Balb/c, female C57BL/6 scid, and female C57BL/6 B-cell deficient (homozygous for the lgh-6^{tmlCgm}) mice (6-8 weeks old), were purchases from Jackson Laboratories (Bar Harbor, Me.) and kept at the animal facility of Genetics Institute according to the Institutes guidelines.

Tumor Models—The following tumro models were used in these studies: a radiation-induced SJL/J acute myloid leukemia (AML) model, a C1598 acute myeloid leukemia model, a B16F1 melanoma model, and a MB49 bladder carcinoma. In the AML model, frozen spleen mononuclear cells isolated from moribund leukemic mice (>95% leukemic cells) were used. The B16F1 and C1498 cell lines were purchased from American Type Culture Collection (ATCC; Rockville, Md.). All tumor cell lines were maintained in vitro at 37° C. in Dulbecco's modified Eagle's medium containing 10% fetal calf serum, 2% glutamine and 1% penicillin-streptomycin. For the establishment of tumors, 10⁵ AML or 2×10⁵ C1498 cells were injected intravenously (IV) in the tail vein of SJL or C57BL/6 mice, respectively, and 10⁵ B16F1 or MB49 cells were injected intradermally (ID) in the flank of C57BL/6 mice. Tumor-bearing animals either died within 20-35 days post tumor-inoculation (AML/C1498), or were sacrificed when tumors reached a size of approximately 400-600 mm² (B16F1/MB49).

Expression of hSDF-1β by Tumor Cells—Manipulations were performed using standard sterile tissue culture technique, and using media and reagents from various commercial suppliers. For the expression of hSDF-1β by tumor cells, PTY67 packaging cell lines secreting replication-defective retroviruses encoding hSDF-1β were used. Original PE501-SDF-1β packaging cell lines were developed by inserting hSDF-1β cDNA into a retroviral vector and transferring the vector into the ecotropic packaging cell line PE501. PE501-SDF-1β packaging cells then secreted murine stem cell virus (MSCV), encoding human SDF-1β. PE501-SDF-1β packaging cells did not grow well in culture. Therefore, amphotropic PT67 cells (Clonetech™) were infected with supernatant from PE501-SDF1β cell lines, and PT67-SDF-1β packaging cells were developed. These PT67-SDF-1β producer clones were easier to grow than PE501-SDF-1 β clones. The retroviral vector backbone utilized the LTR of the murine stem cell virus (MSCV) and contained a selectable neo gene under the control of an encephalomyocarditis virus (EMCV) internal ribosome entry site (IRES). Producer cells secreting mock virus were used for infection of control cells. In some in vivo tumorgenicity experiments, wild-type (wt) instead of control cells were used. Packaging cells were maintained at 37° C. in Dulbecco's modified Eagle's medium containing 10% fetal calf serum, 2% glutamine, 1% penicillin-streptomycin and 1 mg/mL neomycin (G418). For the expression of SDF-1β, tumor cells (5-7×10⁵/mL) were exposed twice to viral (PT67-SDF1β) supernatant for 4-6 hours in the presence of 8 μg/mL polybrene. Infection of tumor cells were performed as follows: wild-type tumor cells (5-7×10⁵/mL) were exposed two or three times to viral supernatant for 4-6 hours in the presene of 8 mg/mL polybrene. Designated numbers of transduced, G418-selected tumor cells (with the exception of the AML SJL model, in which infected AML cells were not selected) were used for in vivo injections.

Human SDF-1β enzyme-linked immunosrbant assay (ELISA)—P67SDF packaging cells and tumor cells (10⁶ cells/mL) were cultured for 24 h and levels of hSDF-1β in culture supernatants were determined by sandwich ELISA. The sensitivity of the assay is 5 pg/mL.

⁵¹C Release CTL Assays—Spleens were collected from mice 11 weeks after SDF-B16F1 tumor inoculation/rejection and single-cell suspensions were prepared. Splenocytes (5×10⁶) were co-cultured with irradiated (7335 cGy) B16F1 (1×10⁵) in 2 mL of complete RPMI/well of a 24-well tissue culture plate. Six days later, splenocytes were harvested and used as effector cells in cytotoxic T lymphocyte (CTL) assays. B16F1 (H-2^{d}) or control allogenic TSA (H-2^{b}) tumor cells (2×10⁶) were labeled with 200 μCi of ⁵¹Cr for 90 minutes, washed twice, and used as targets (5000/well) in CTL assays. The standard 4-h CTL assays were set up with various effector to target (E/T) ratios.

In vivo T Cell Subset Depletions—The monoclonal antibodies (mAb) GK1.5 (rat anti-mouse CD4) and 53-6.7 (rat anti-mouse CD8) were used for in vivo T cell subset depletions. The mAbs were produced and purified by standard techniques at Genetics Institute, Inc. For in vivo depletion experiments, the mice were injected intraperitoneally (IP) on three consecutive days with mAb (0.5 mg/injection). Depletion of CD4⁺ or CD8⁺ T cells was verified 3 days after the last injection by flow cytometric analysis of spleen cells. The analysis showed that >95% depletion of the appropriate subset was achieved with normal levels of the other subset (data not shown). Three days after the last injection the mice were injected IV with live SDF-C1498 cells and antibody injection continued every 5 days for 3 weeks.

Immunohistochemistry—C57BL/6 mice were injected ID with 10⁶ live wild-type SDF-B16F1 cells. Tissues were then collected on days 3, 7, and 14 (10 mice/time point/cell type) after tumor inoculation, and tumor cell and immune infiltrates (ICI) were evaluated. Tissues were bisected and one half were cryopreserved in O.C.T. by liquid nitrogen-cooled isopentane method, and the other half fixed in 10% neutral-buffered formalin. For histological evaluation, 5 μm sections from paraffin embedded tissues were stained with hematoxylin and eosin (H+E). For CD4 (L3T4), CD8a (53-6.7) and Gr-1 (RB6-8C50) immunohistochemistry, cryopreserved samples were cryosectioned onto capillary gap micro slides and fixed in acetone before storing at −20° C. Immediately prior to staining, stored cryosections were fixed in cold acetone for 5 minutes, air-dried, blocked with avidin/biotin block (Zymed Laboratories, Inc.) and finally washed in PBS. For paraffin embedded tissues, either Proteinase K or microwave antigen retrieval was used. For CD45R/B220 (RA3-6B2, Pharmingen), Proteinase K (Sigma) was used as the antigen retrieval solution. Slides were treated with Proteinase K at 25 μg/mL for 15 minutes in a 37° C. incubator. For CD3 (CD3-12; Serotec) immunolabeling, microwave antigen retrieval technique was used prior to the incubation of 10 μg/mL of CD3 primary antibody. The paraffin sections were deparaffinized, rehydrated with water and placed in 0.01M citrate buffer, pH 6.0. The sections were heated to 99° C. for 5 minutes and allowed to cool for 5 minutes, followed by re-heating to 99° C. for 5 minutes and subsequent cooling for a further 2 minutes before rinsing in distilled water. The sections were washed in phosphate buffered saline (PBS), pH 7.4 for 2 minutes before being assembled and immunolabelled on an automated immunostainer (Tech Mate 500, Bentana). Serial sections were stained with rat immunoglobulins as negative controls. An indirect streptavidin-peroxidase method was used with DAB (3,3′ diaminobenzidine tetrahydrochloride) as color chromogen.

Proliferation Assays—Spleens were harvested from C57BL/6 mice and single-cell suspensions were prepared. For T cell enrichment, mouse T cell enrichment columns (R&D Systems) were used according to the manufacturer's instructions. The purity of CD3⁺ T cells after column elution was 85%-87%. Splenocytes or T cells (2×10⁵ cells/well) were cultured in flat-bottomed 96-well plates with sub-optimal dose (500 ng/mL) of anti-CD3 mAb 145-2C11 (with or without 200 ng/mL rhSDF-1β [PharMingen]) and increasing numbers of irradiated (4000 cGy) SDF-1β-tumor or control tumor cells. Response to costimulation with anti-CD3 plus anti-CD28 (5 μg/mL) antibodies was used as a positive control. Proliferation of responder cells was measured after 72 hours by the incorporation of ³H-thymidine (1 μCi/well) for the last 6-9 hours of incubation.

Statistical Analysis—Individual experiments consisted of 10 mice/treatment group. The staistical survival analysis was performed using the standard Mantel-Cox logrank test. Proliferation results were mean±SD. The statistical significance between various groups was analyzed using the Student's t-test.

### Example II

This example illustrates the modification of tumor cells to secrete and express an increased amount of hSDF-1β.

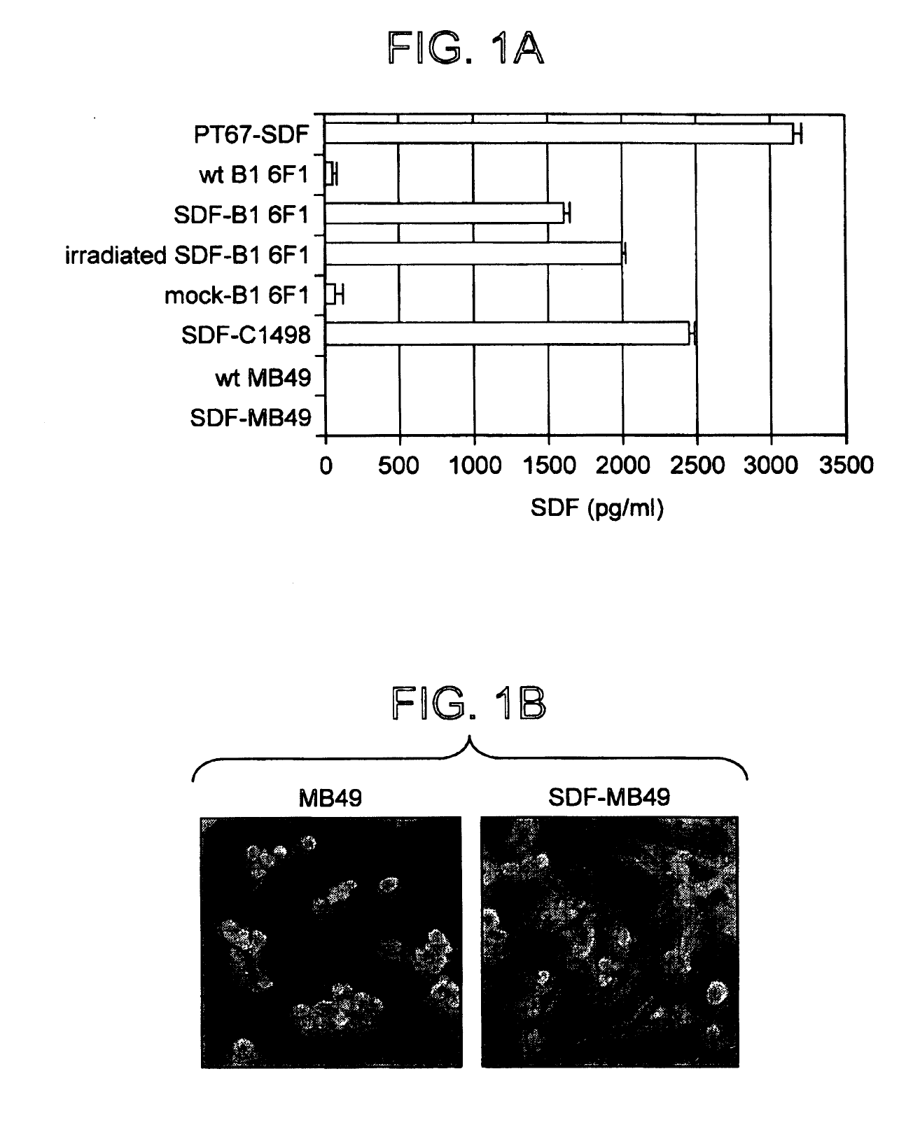

Tumor cells were transduced with PT67-hSDF1β or PT67-mock retroviral supernatants as described in Example I. Cell lines were cultured for 24 hours and supernatants were collected and assayed for hSDF-1β levels as determined by hSDF-1β ELISA (FIG. 1a). The in vitro growth rate characteristics of SDF-1β-tumor cells was similar to the growth rate of control (mock-transduced) cells (data not shown). Immunostaining and flow cytometric analysis revealed enhanced LFA-1 (CD11a/CD18) expression by SDF-1β-tumor cells as compared to control cells, but no differences in expression of CD80, CD86, or MHC class I when SDF-1β-tumor and control cells were compared (data not shown). Expression of SDF-1 by the MB49 and B16F1 cells resulted in apparent morphological changes in the form of long cytoplasmic projections and greater adherence to plastic (FIG. 1b).

### Example III

This example illustrates the antitumor activity of SDF-1β in four in vivo systemic hematological malignancy models. For in vivo tumorigenicity studies, mice were injected intravenously with live control (wild-type or mock-infected) cells or hSDF-1β transduced cells, and their clinical outcome was subsequently assessed.

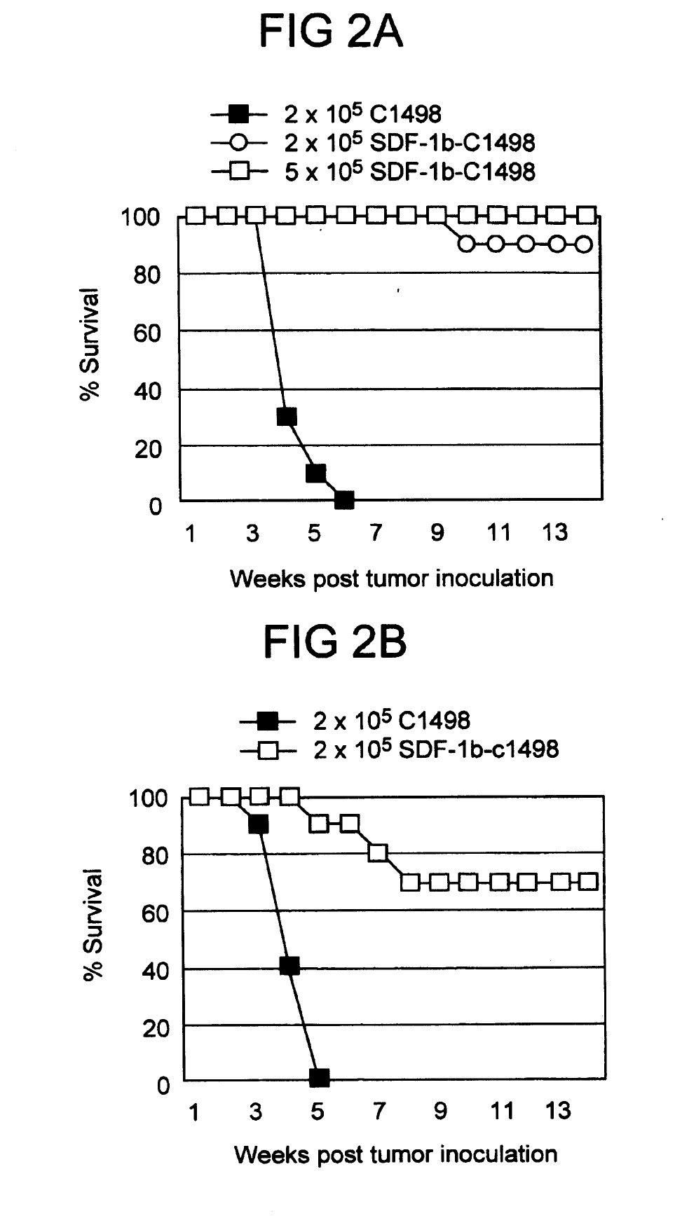

C1498 acute myeloid leukemia cells were transduced with supernatant from the PT67-SDF-1β producer cell line described in Example I to generate SDF-1β-C1498 cells. C57BL/6 mice were injected intraveneously with 2×10⁵ control cells (C1498 cells), 2×10⁵ SDF-1β-C1498 cells or 5×10⁵ SDF-1β-C1498 cells and the percent survival was determined at the times indicated in FIG. 2 (weeks post tumor inoculation). Approximately 70-90% of animals injected with 2×10⁵ SDF-1β-C1498 cells survived past 13 weeks and approximately all animals injected with 5×10⁵ SDF1β-C1498 cells survived past 13 weeks.

C57BL/6 mice that had rejected live SDF-C1498 cells were challenged 3 or 4 months later with live wild type C1498 cells. Naive C57BL/6 mice injected with live wild type C1498 cells were used as controls. At least 40% of mice challenged 3 months later with live wild type C1498 cells survived at least 50 days post challenge. At least 60% of mice challenged 4 months later with live wild type C1498 cells survived at least 50 days post challenge (data not shown).

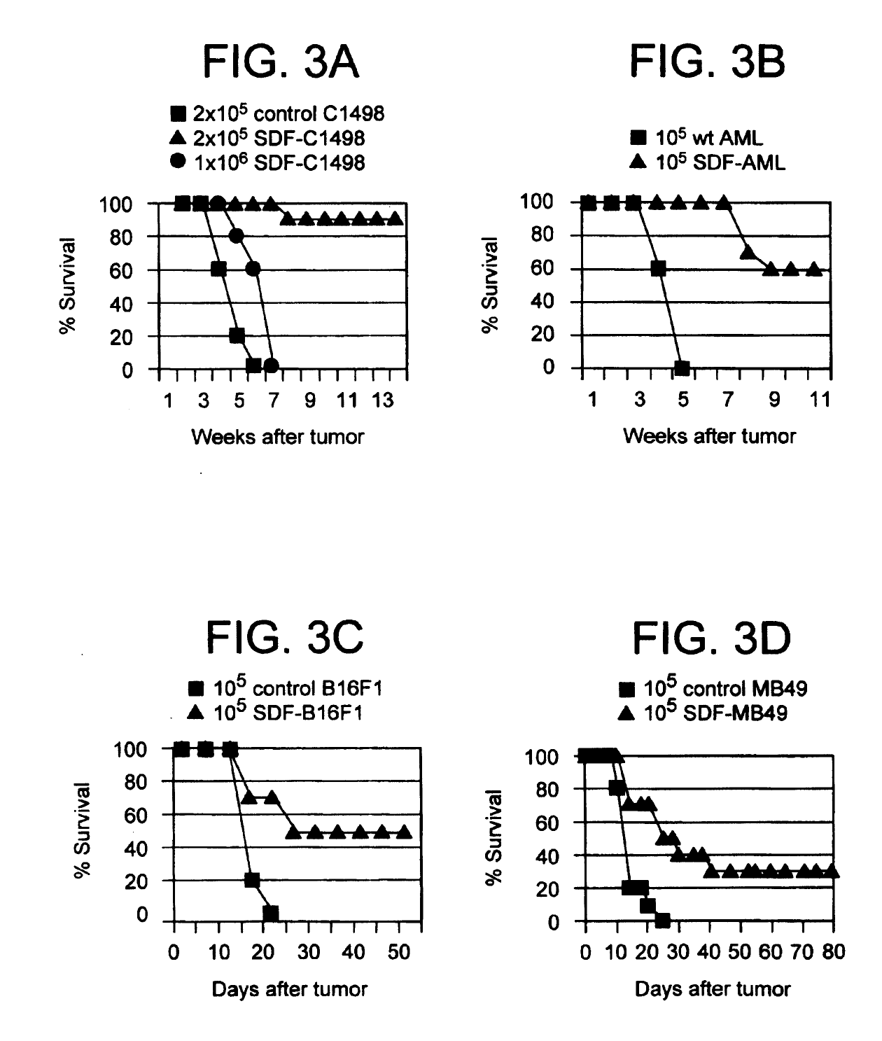

This experiment was repeated and expanded to include several other tumor models. In these studies, C57BL/6 mice (10 mice/group) were injected IV with the indicated numbers of live SDF-C1498 cells or control C1498 cells. In several experiments, injection of C57BL/6 mice with 2×10⁵ live SDF-C1498 cells resulted in 90%-100% tumor rejection. However, mice injected with 1×10⁶ SDF-C1498 (●) had delayed tumor growth (P<0.01 versus control (▪) mice), but eventually developed lethal leukemia; 90% of the mice injected with 10×10⁵ SDF-C1498 cells (▴) rejected their leukemia (P<0.0001 versus control mice) (FIG. 3a). Eventually all of these mice developed lethal leukemia (FIG. 3a). This graph is representative of 4 independent experiments. In the SJL-AML model (FIG. 3b), SJL/J mice (10 mice/group) were injected IV with 105 wild-type AML (▪) of SDF-AML (▴) cells. All mice (100%) of the mice had significant delay in leukemia growth and 60% of the mice rejected the leukemia cells, whereas all control mice developed lethal leukemia (P<0.005) (FIG. 3b). This graph is representative of 2 independent experiments. In the B16F1 model (FIG. 3c), C57BL/6 mice (10 mice/group) were injected ID in the flank with control B16F1 (▪) or SDF-B16F1 (▴) cells. All control mice developed lethal tumors, while 50%-60% of mice injected with SDF-B16F1 cells reproducible showed delayed tumor growth, but ultimately developed lethal tumors. In this model, although some mice (10% to 20%) had eventually small palpable tumors, these tumors did not progress, and eventually regressed. Thus, as shown in FIG. 3c, 50% of mice injected with SDF-B16F1 cells had long-term, tumor-free survival (P<0.005). This graph is representative of 3 independent experiments. In the MB49 model (FIG. 3d), C57BL/6 mice (10 mice/group) were injected ID in the flank with control MB49 (▪) or SDF-1β-MB49 (▴) cells. Although SDF-1β levels secreted by transduced MB49 cells were below the sensitivity of the ELISA, the tumorgenicity of SDF-1β-MB49 cells was reproducibly lower (P<0.005) than control cells (FIG. 3d). This graph is representative of 2 independent experiments. In all experiments, mice that has rejected their tumors did not develop any clinical signs of toicity and remained above and tumor-free for several weeks or months after tumor inoculation.

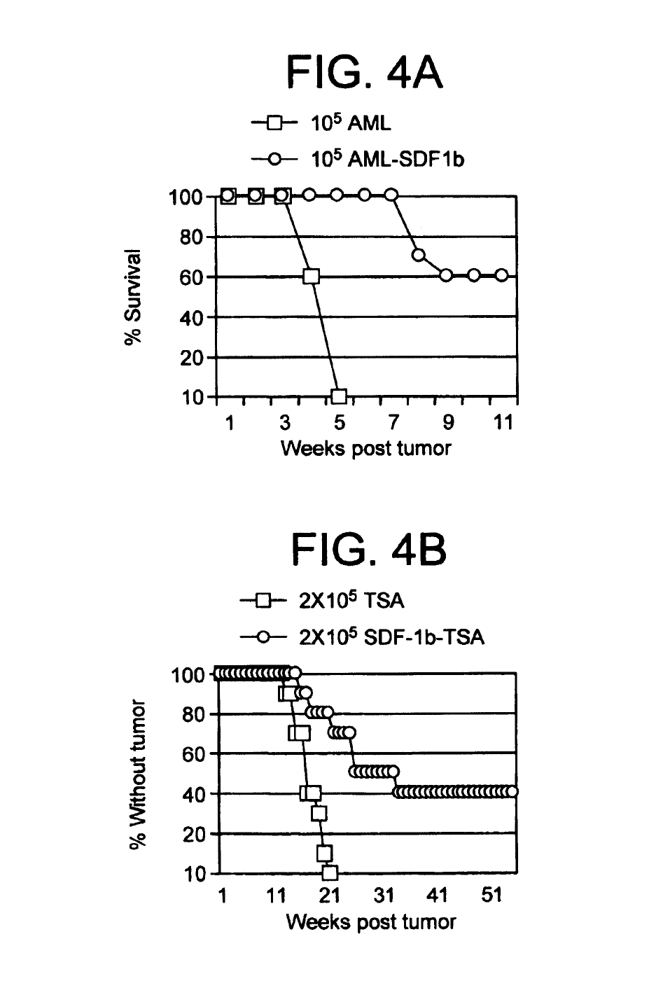

Acute myeloid leukemia (AML) cells were transduced with suprenatant from the PT67-SDF-1β producer cell line described in Example I to generate SDF-1β-AML cells. SJL mice were injected IV with 1×10⁵ AML cells or 1×105 SDF-1β-AML cells and % survival was determined at the times indicated in FIG. 4 (weeks post tumor inoculation). All animals injected with 2×10⁵ SDF-1β-AML cells survived past 7 weeks and approximately 60% of animals injected with 2×10⁵ SDF-1β-AML cells survived past 13 weeks.

TSA cells (mammary adenocarcinoma cells) were transduced with supernatant from the PT67-SDF-1β producer cell line described in Example I to generate SDF-1β-TSA cells. Balb/c mice were injected subcutaneously with 2×10⁵ TSA cells or 2×10⁵ SDF-1β-TSA cells and % survival was determined the times indicated in FIG. 4 (weeks post tumor inoculation). All animals injected with 2×10⁵ SDF-1β-TSA cells survived past 11 days and approximately 40% of animals injected with 2×10⁵ SDF-1β-TSA cells survived past 51 days.

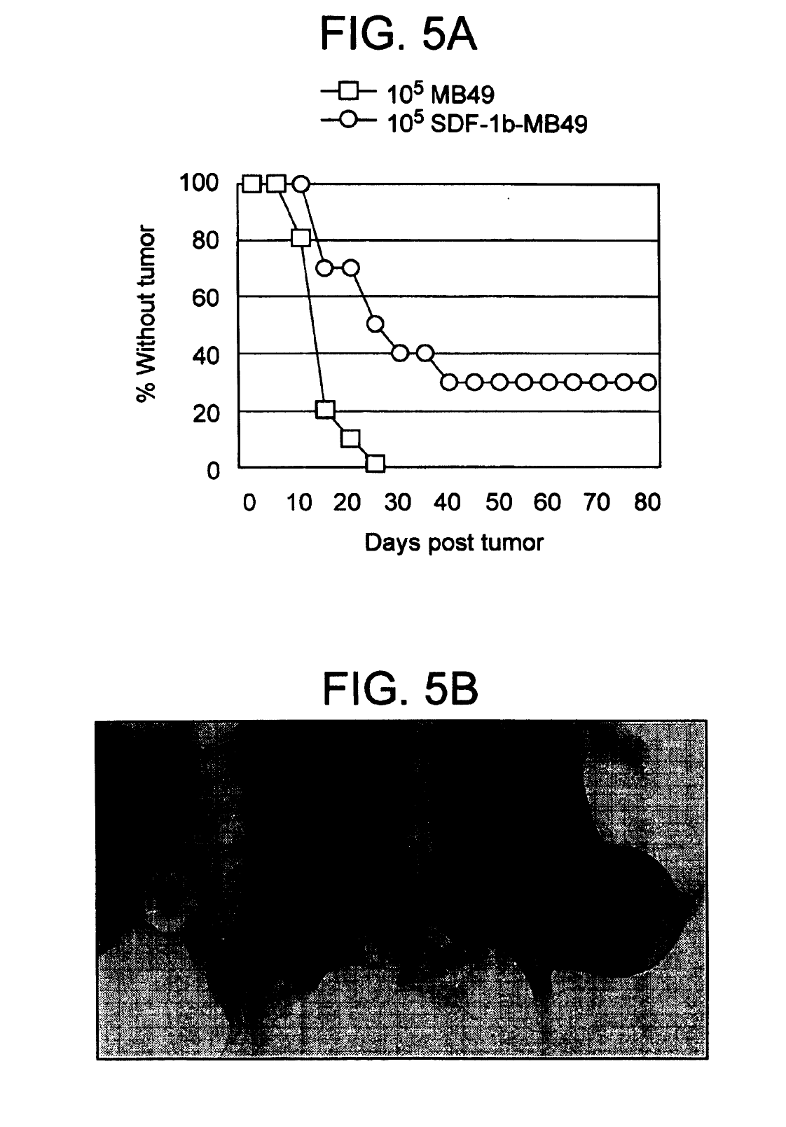

C57BL/6 mice were injected subcutaneously with 1×10⁵ wild-type MB49 cells (bladder carcinoma cells) or with 1×10⁵ SDF-1β-MB49 cells. Levels of SDF-1β secreted by the SDF-1β-MB49 were below the sensitivity of SDF-1β ELISA utilized. Small tumor masses developed in the SDF-1β-MB49 injected mice, however, at least 30% of mice injected with SDF-1β-MB49 cells were without tumors at least 80 days post injection. By contrast, all mice injected with wild-type MB49 cells had tumors by 30 days post injection (FIGS. 5A-B).

### Example IV

This example illustrates that irradiated SDF-1β-tumor cells support the induction of systemic and therapeutic immunity.

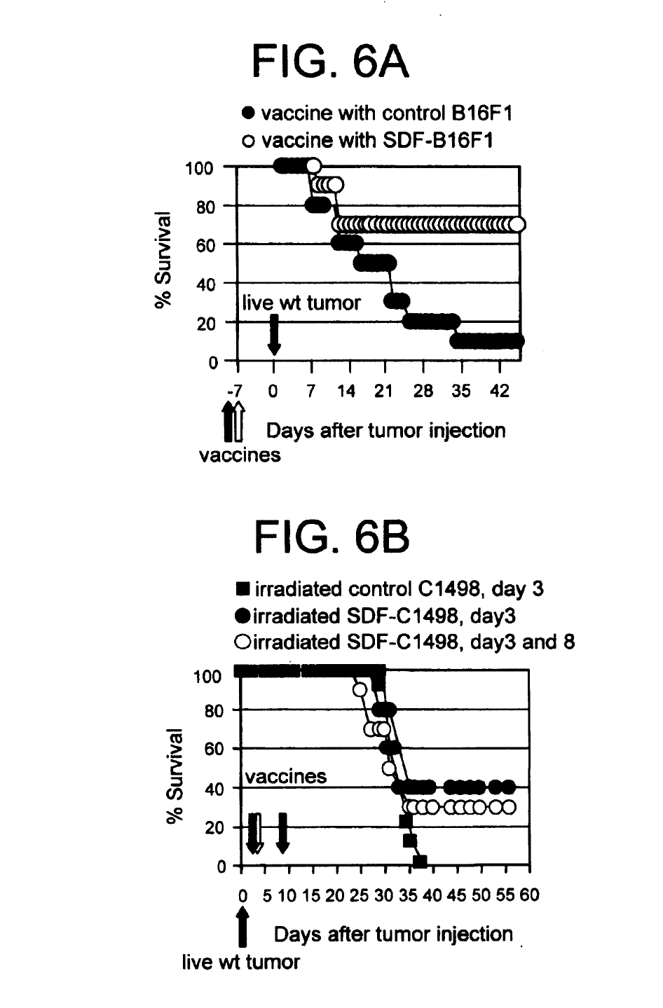

C57BL/6 mice (10 mice/group) were vaccinated ID in one flank with irradiated 10⁵ SDF-B16F1 (o) or control B16F1 cells (●) and challenged a week later in the opposite flank with live 10⁵ wild-type B16F1 cells. Vaccination with SDF-1β-B16F1 cells resulted in 70% protection of the mice and resistance to wild-type tumor challenge (P=0.018 versus control wild-type vaccines); vaccination with wild-type B16F1 only protected 10% of the mice (FIG. 6a). This graph is representative of 2 independent experiments.

In another experiment, C57BL/6 mice (10 mice/group) were injected IV with live 2×10⁵ wild-type C1498 cells. On day three, they were immunized IV with irradiated 10⁵ control (▪) or SDF-1β-C1498 cells (●). A third group of mice were immunized twice (day 3 and 8 post live wild-type tumor inoculation) with 10⁵ SDF-1β-C1498 cells (o). Day three immunization cured 40% of the leukemic mice (P=0.09 versus control), day 3 and 8 immunizations cured 30% of leukemic mice (P=0.49 versus control) (FIG. 6b). These results were replicated in a second experiment.

### Example V

This example elucidates the mechanism by which SDF-1β-transduced tumor cells effect anti-tumor immune responses and tumor rejection.

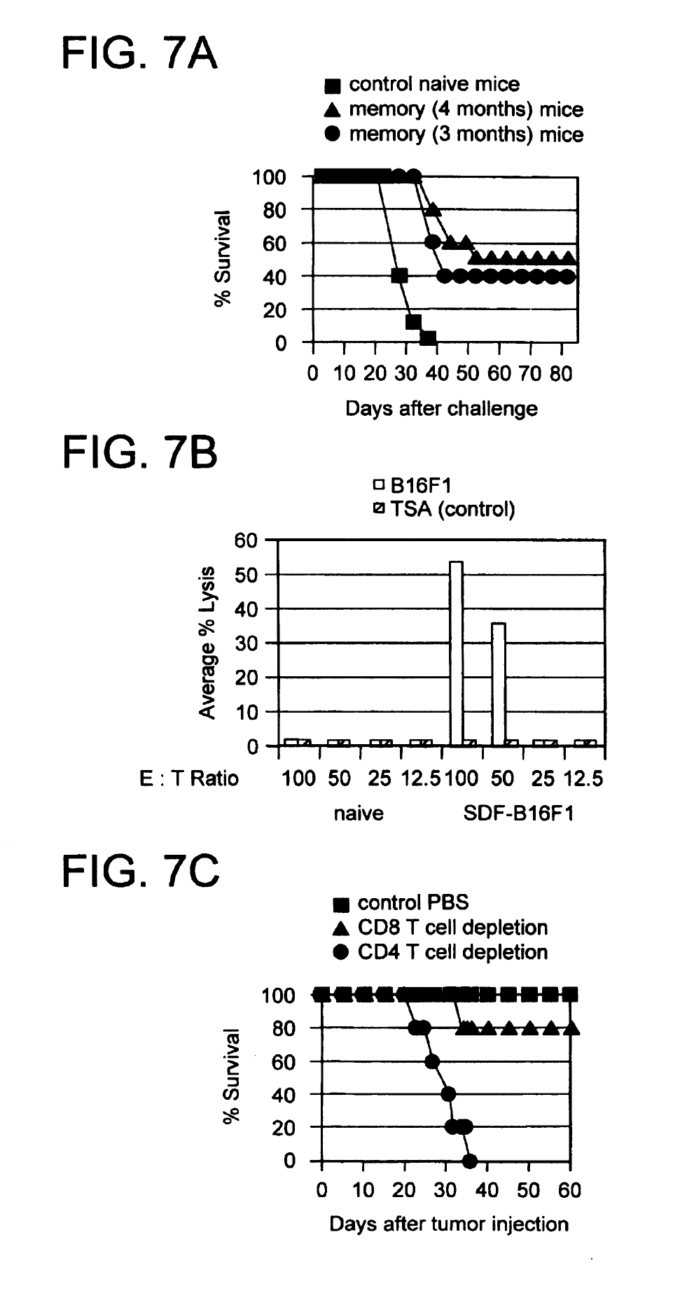

In this experiment, C57BL/6 mice (10 mice/group) were challenged IV with 10⁵ wild-type C1498 cells 3 months (●) or 4 months (▴) after the rejection of live SDF-1β-C1498 cells. Naïve C57BL/6 mice were used as controls (▪). Both groups had delayed tumor growth, as compared to control animals, and 40% (3 months) and 50% (4 months) of the mice that rejected SDF-1β-secreting tumors had generated a sufficient memory response to resist this tumor challenge (P<0.0001 versus control mice) (FIG. 7a).

In additional experiments, splenocytes from C57BL/6 mice that had rejected live SDF-B16F1 cells were assayed 3 months later for in vitro CTL activity against wild-type B16F1 cells or control TSA cells utilizing the ⁵¹Cr release CTL assay as described in Example I. Specifically, spleens were collected from mice 11 weeks after SDF-1β-B16F1 tumor inoculation/rejection and splenocytes were co-cultured with irradiated B16F1 (H-2^{d}) or control allogeneic TSA (H-2^{b}) tumor cells which were used as targets in the standard 4-h CTL assays. CTL activity was detected in splenocytes from C57BL/6 mice that had rejected live SDF-B16F1 cells but not in splenocytes from control naive mice (FIG. 7b). However, splenocytes from C57BL/6 mice that had rejected live SDF-1β-B16F1 cells had no cytoxic activity when assayed against control TSA cells (FIG. 7b). The results are representative of two independent experiments.

In another experiment, CD4⁺ T cells were shown to be indespensible for SDF-1β-mediated tumor rejection. C57BL/6 mice were depleted of CD4⁺ (●) or CD8⁺ (▴) T cells, as described in Example I. Control mice were treated with PBS (▪). The results showed that 100% of the mice treated with PBS and 80% of the mice treated with anti-CD8⁺ mAb rejected the SDF-1β-C1498 cells and did not develop any signs of leukemia (FIG. 7c). Depletion of CD4⁺ T cells completely abrogated the immune mechanisms leading to SDF-1β-C1498 rejection and 100% of the mice developed lethal leukemia (P<0.0001 versus control PBS).

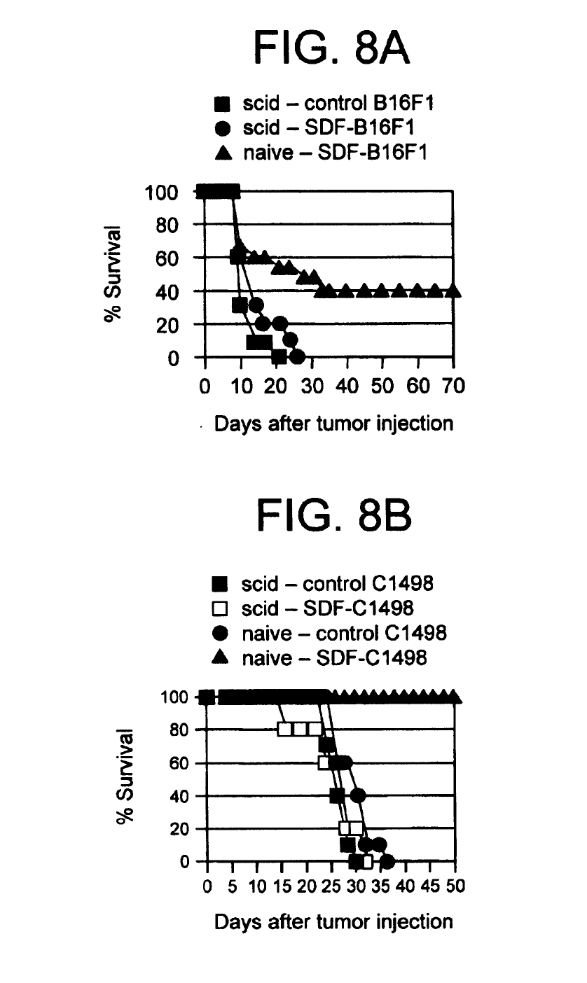

To investigate the mechanism of SDF-1β-induced tumor immunity, scid mice were injected with SDF-1β-B16F1 cells. As controls, normal mice were injected with either wild-type B16F1 cells or SDF-1β-B16F1 cells. The percent survival was determined at the indicated days post tumor inoculation. Whereas at least 40% of normal mice injected with SDF-1β-B16F1 cells survived at least 73 days post inoculation, no normal mice inoculated with wild-type B16F1 cells survived past 21 days and scid mice injected with SDF-1β-B16F1 cells did not survive past 25 days (FIG. 8a). These data indicate that rejection of SDF-1β-B16F1 cells is T cell dependent.

In another experiment, the pattern of in vivo SDF-1β-tumor growth in immunodeficient mice was assessed. Specifically, C57BL/6 scid mice (10 mice/group) were injected IV with 10⁵ control C1498 (▪) or SDF-1β-C1498 (X) cells. Naïve mice were injected with 10⁵ control C1498 (●) or SDF-1β-C1498 (▴) cells and were used as a control. In both tumor models, SDF-1β-transduced and control cells grew in all scid animals (FIG. 8b). The results are representative of two separate experiments. Furthermore, because SDF-1β was originally described as B cell growth factor, the growth of SDF-1β-tumor cells in B-cell deficient mice was also tested. Results demonstrated that 70% to 80% of B-cell deficient mice injected with either control or SDF-1β-B16F1 cells rejected their tumors (data not shown).

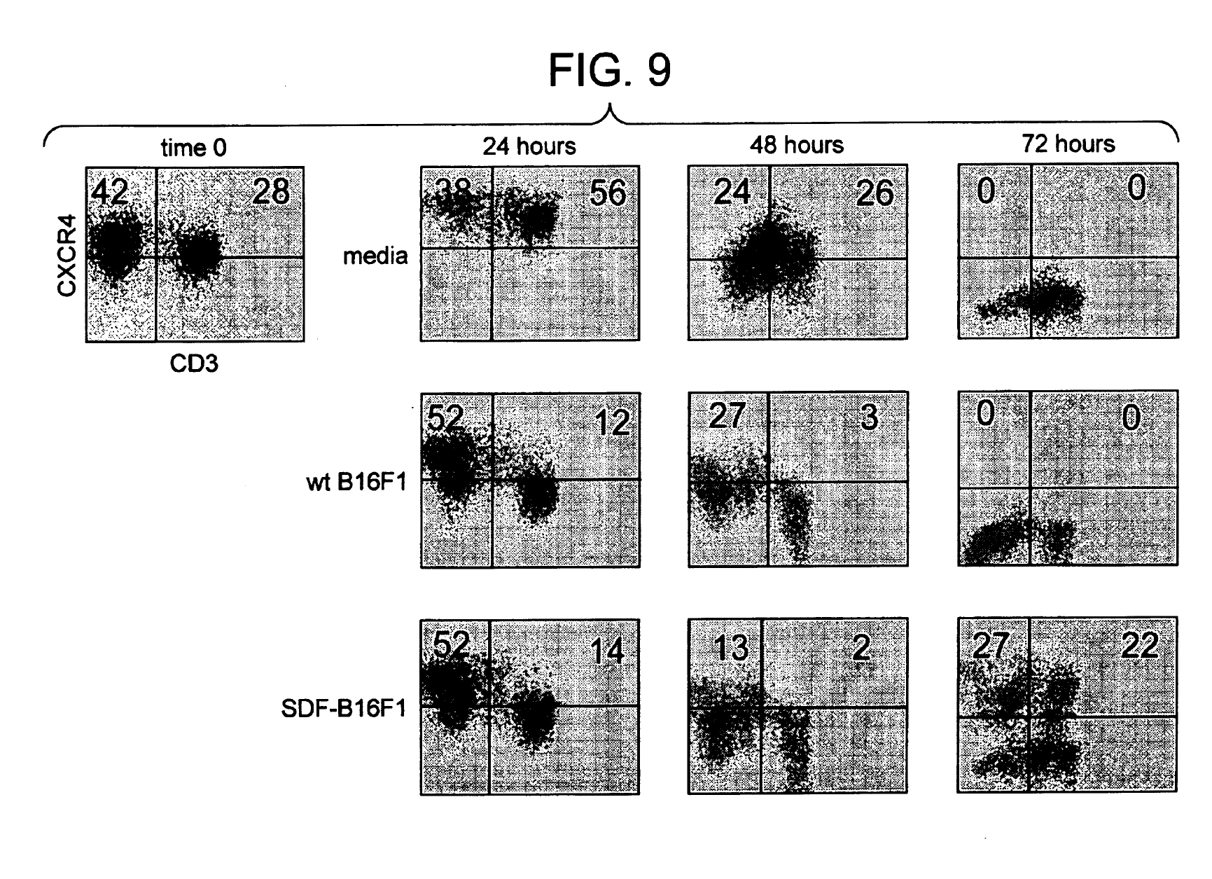

To further characterize the immune cells that participate in tumor rejection, a series of histology/immunohistochemistry studies during the in vivo growth of wild-type B16F1 and SDF-1β-B16F1, as described in Example I, were performed. C57BL/6 mice were injected ID with wild-type B16F1 or SDF-1β-B16F1 cells and tissues collected for examination. As shown in FIG. 9, prominent immune cell infiltrates (CD3, CD4 and CD8 T cells) were observed in SDF-1β-tumors, but not in wild-type tumors (original magnification 10×). No major differences in other cell types (B cells, neutrophils, macrophages) could be identified (data not shown). On day 14, all control animals but few animals with SDF-1β tumors has palpable tumors. Histologically, wild-type animals had very large tumors consisting of numerous tumor cells without ICI, whereas most SDF-1β animals had only scattered tumor cells or small cell clusters and significant ICI (dat not shown). Interestingly, inflammatory cells (macrophages and netrophils) were delectable in both groups with fewer cells found in the control animals (data not shown).

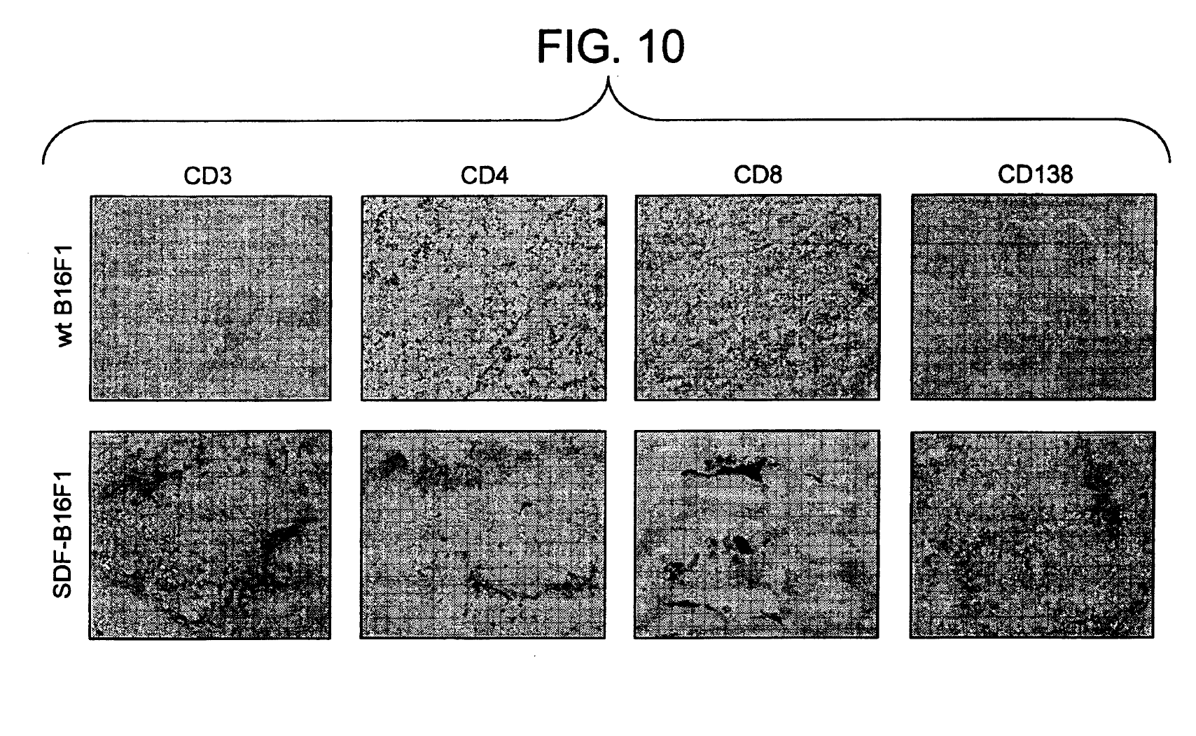

Next, experiments were performed to determine the effect of SDF-1β expression on the SDF-1β receptor on immune cells. Spleens were harvested from C57BL/6 mice and single cell suspensions were prepared. Splenocytes (2×10⁶ cells/mL) were cultured in media alone, or with wild-type B16F1, or SDF-1β-B16F1 tumor cells (splenocyte:tumor cell ratio 3:1). At time points 24 h, 48 h, and 72 h, splenocytes were harvested, washed with cold PBS and stained for CD3 and CXCR4 (SDF-1β receptor) expression. The following antibodies were used for flow cytometry studies: FITCconjugated monoclonal antibody (MoAb) CD3e (145-2C11) (PharMingen, San Diego, Calif.) and the human SDF-Fc fusion protein (Genetics Institute). The Fc portion of the SDF-Fc fusion protein is a human IgG4. After staining, the cells were fixed in 1% paraformaldehyde and analyzed on a FACScan™ flow cytometer. (FIG. 10). These data demonstrate that SDF-1β-B16F1 cells are capable of restoring CXCR4 expression on murine splenocytes.

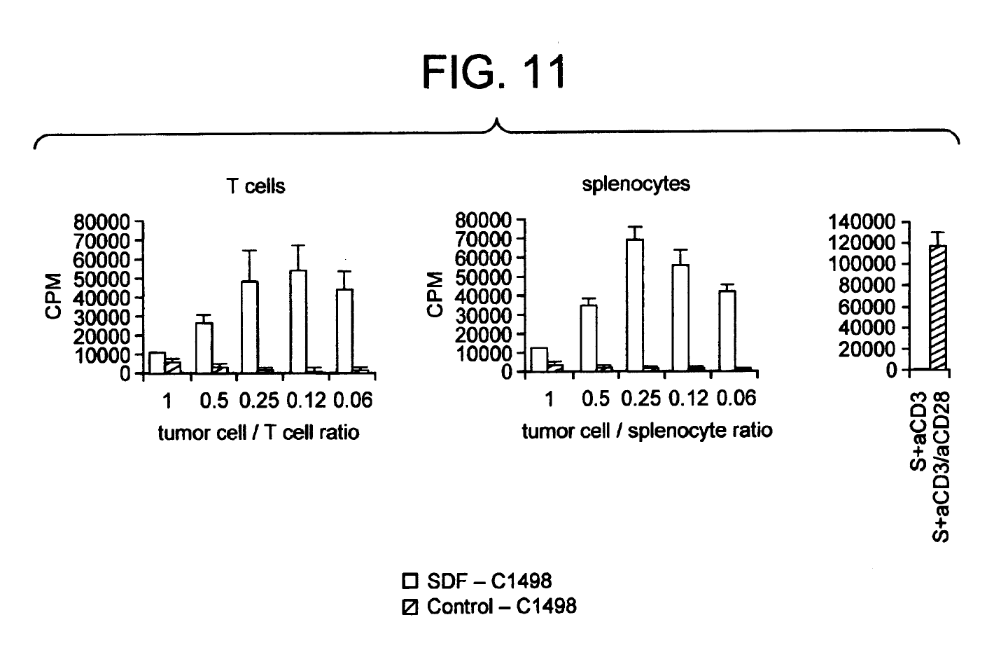

Finally, the in vitro T cell costimulatory activity of SDF-cells and control cells in the C1498 and B16F1 models were compared. C57BL/6 splenocytes or enriched T cells were co-cultured with irradiated SDF-1β-C1498 or control C1498 cells at the indicated stimulator/responder ratios as described in Example I. The proliferative responses of splenocytes to anti-CD3 (S+αCD3) and to anti-CD3/anti-CD28 (S+CD3/αCD28) were used as controls. Proliferation of responder cells was measured after 72 hours by the incorporation of ³H-thymidine (1 μCi/well) for the last 6-9 hours of incubation. As shown in FIG. 11, profound prolifeative responses were observed when T cells or splenocytes were co-cultured with irradiated SDF-1β-C1498 cells. In contrast, proliferative responses were almost completely absent in cultures with control cells. However, when cells in control cultures were incubated in medium supplemented with recombinant SDF-1β, proliferation of T cells was restored (data not shown).

Conclusion:

The above-described results demonstrate: (i) gradual continuous release of low doses of SDF-1β by SDF-1β-transduced tumor cells results in effective anti-tumor responses and tumor rejection; (ii) mice that had previously rejected live SDF-1β-tumor cells, develop long-lasting memory cells, are immune to rechallange with live wild-type tumor cells, and have tumor specific CTL activity; (iii) mice that are previously immunized with irradiated SDF-1β transduced tumor cells in one flank, are protected against inoculation in the opposite flank with live wild-type tumor cells; (iv) in vitro cultures suggest that secretion of SDF-1β by SDF-1β-tumor cells may restore irreversible CXCR4 down-regulation mediated by wild type tumor cells, thus enhancing chemoattraction of immune cells to the tumor site; and (v) SDF-1β-tumor cells were not rejected by scid mice. Moreover, histology showed heavy cellular infiltrates with immune cells surrounding tumor masses that secrete SDF-1β.

Equivalents

Those skilled in the art will recognize, or be able to ascertain using no more than routine experimentation, many equivalents to the specific embodiments of the invention described herein. Such equivalents are intended to be encompassed by the following claims.

What is claimed:

1. A vaccine comprising tumor cells isolated from a subject which have been modified to secrete an increased level of SDF-1β relative to unmodified tumor cells, wherein said vaccine confers tumor immunity upon administration to said subject.

2. The vaccine of claim 1, wherein modifying said tumor cells comprises transducing said cells with a nucleic acid molecule which encodes SDF-1β.

3. The vaccine of claim 2, wherein said nucleic acid molecule which encodes SDF-1β is in the form of a vector.

4. The vaccine of claim 3, wherein the vector is a recombinant expression vector.

5. The vaccine of claim 3, wherein the recombinant expression vector is selected a viral expression vector.

6. The vaccine of claim 4, wherein the recombinant expression vector is a replication-defective retroviral vector.

7. The vaccine of claim 2, wherein said modified tumor cells have been expanded in culture prior to introduction of said nucleic acid molecule which expresses SDF-1β.

8. The vaccine of claim 1, further comprising a pharmaceutically acceptable carrier.

9. A method for producing an autologous tumor vaccine comprising:
(a) isolating tumor cells from a subject leaving cancer; and
(b) modifying said tumor cells such that they secrete an increased level of SDF-1β relative to unmodified tumor cells;
such that an autologous tumor vaccine is produced.

10. The method of claim 9, wherein said cells to be modified are isolated from a tumor which has been surgically removed from said subject.

11. The method of claim 9, wherein said cells to be modified are isolated from a biopsy of a tumor in said subject.

12. The method of claim 9, wherein said cells to be modified are expanded in culture prior to modification of said cells.

13. A method for treating a subject having cancer comprising administering to said subject the autologous tumor vaccine of claim 1 in an amount sufficient to inhibit tumor growth, such that said subject is treated.

14. The method of claim 13, wherein the autologous tumor vaccine is administered when the tumor burden of said subject is low.

15. The method of claim 13, wherein the autologous tumor vaccine is administered after said subject has undergone chemotherapy.

16. The method of claim 13, wherein the autologous tumor vaccine is administered after said subject has undergone radiation therapy.

17. The method of claim 13, further comprising monitoring the antitumor immune response in said subject

18. The method of claim 13, wherein the cells of said tumor vaccine are irradiated prior to administration to said subject.

19. The method of claim 13, wherein the cells of said tumor vaccine are admixed with an adjuvant prior to administration.

20. The method of claim 13, wherein said tumor vaccine is administered at or near at least one site of a tumor in said subject.

21. The method of claim 13, wherein said tumor vaccine is administered at or near at least one site from which a tumor has been surgically removed from said subject.

22. A method for promoting an antitumor response in a subject having cancer comprising administering to said subject the autologous tumor vaccine of claim 1, such that said subject develops an antitumor response to said vaccine.

23. The method of claim 22, wherein the autologous tumor vaccine is administered when the tumor burden of said subject is low.

24. The method of claim 22, wherein the autologous tumor vaccine is administered after said subject has undergone chemotherapy.

25. The method of claim 22, wherein the autologous tumor vaccine is administered after said subject has undergone radiation therapy.

26. The method of claim 22, further comprising monitoring the antitumor immune response in said subject

27. The method of claim 22, wherein the cells of said tumor vaccine are irradiated prior to administration to said subject.

28. The method of claim 22, wherein the cells of said tumor vaccine are admixed with an adjuvant prior to administration.

29. The method of claim 22, wherein said tumor vaccine is administered at or near at least one site of a tumor in said subject.

30. The method of claim 22, wherein said tumor vaccine is administered at or near at least one site from which a tumor has been surgically removed from said subject.
